SEQUENCE LISTING (8 sequences)

SEQ ID NO: 1 - DNA, 3269 bp, Homo sapiens
cacttccggcctcgcgagggccgcaatcactgctccgcagttcccgcctgcattcctcgcgccgtcttcctggagtcccagctctccttcagcccgccccaacgctgacgctcagtcctcaggcgtcgagggtagctcctgtgaggggctcgcttggcgcacgcaaaacgctcagcgcgcaccacagggcgtccgccccaaccccgcccccggaggcctccagctcggccccgcccctgtcccttccccgtcgcggaggcagcctagcctcgcgccccgcccgttgcttctgccctccggccttcccgccgccgtcgccgggaccagccgctcggggccgggctgatacagccgcttcaccgtgcccctgcccgcgaccatggcctcctccgaggtggcgcggcacctgctctttcagtctcacatggcaacgaaaacaacttgtatgtcttcacaaggatcagatgatgaacagataaaaagagaaaacattcgttcgttgactatgtctggccatgttggttttgagagtttgcctgatcagctggtgaacagatccattcagcaaggtttctgctttaatattctctgtgtgggggaaactggaattggaaaatcaacactgattgacacattgtttaatactaattttgaagactatgaatcctcacatttttgcccaaatgttaaacttaaagctcagacatatgaactccaggaaagtaatgttcaattgaaattgaccattgtgaatacagtgggatttggtgaccaaataaataaagaagagagctaccaaccaatagttgactacatagatgctcagtttgaggcctatctccaagaagaactgaagattaagcgttctctctttacctaccatgattctcgcatccatgtgtgtctctacttcatttcaccgacaggccactctctgaagacacttgatctcttaaccatgaagaaccttgacagcaaggtaaacattataccagtgattgccaaagcagatacggtttctaaaactgaattacagaagtttaagatcaagctcatgagtgaattggtcagcaatggcgtccagatataccagttcccaacggatgatgacactattgctaaggtcaacgctgcaatgaatggacagttgccgtttgctgttgtgggaagtatggatgaggtaaaagtcggaaacaagatggtcaaagctcgccagtacccttggggtgttgtacaagtggaaaatgaaaaccactgtgactttgtaaagctgcgggaaatgctcatttgtacaaatatggaggacctgcgagagcagacccataccaggcactatgagctttacaggcgctgcaaactggaggaaatgggctttacagatgtgggcccagaaaacaagccagtcagtgttcaagagacctatgaagccaaaagacatgagttccatggtgaacgtcagaggaaggaagaagaaatgaaacagatgtttgtgcagcgagtaaaggagaaagaagccatattgaaagaagctgagagagagctacaggccaaatttgagcaccttaagagacttcaccaagaagagagaatgaagcttgaagaaaagagaagacttttggaagaagaaataattgctttctctaaaaagaaagctacctccgagatatttcacagccagtcctttctggcaacaggcagcaacctgaggaaggacaaggaccgtaagaactccaattttttgtaaaacagaagttccagagcacagaaggtcatcatcacaagcaaactttattaaaaaaaaactagaagtgtgctttgattttgctgttatttgttttatcacttctatatttggtgaacagccacagttactgatatttatggaaaagtactttcaagtacaaggtcaatacataagccagagtgaatgatactacaagttgagcatctctaattcaaaaatctgaaatccagaagcttcaaaatctgaatctttttgagcactgacttgaccccacaagtggaaaattccccacccgacacctttgctttctgatggttcagtttaaacagattttgtttcttgcacaaaatttttgtataaattactttcaggctatatgtataaggtggatgtgaaacatgaattatgtaattagagtcgggtcccgttgtgtatatgcagatattccaaacctgaaatccaaaacacttctggtccctagcattttggataagggatactcagcttgtacctatatattcatatatattcactgttgttagaaatgtttaagttgctgttctgtgatgaatctaaatcttttctcttgctaccaagctattgtcactgcagtgcattataccaaagagcgaagtcagtgccactgaaaatacagaacccattaatatcgtggctatctgattacatttatattccaagatgaaccttttttatatatgctaaaaattttggggaatatgttttgggatgtattatggagctaaaactctaacctcttaatagttttatagaacttaaaaattttttatacaattacccaattggtgatatgatcttaagcttttgtgtcagattatttaatatgatgacttcatgctttattatgccttattatggctgacgtattactgtggtgaaacaaaatatctttaaaagttaaaacatccagatatataagctattttttcctaaggataaagtacctttgagcatgagtgtatcacagctttcattaggaaaacttttcattacatacttgtttaaactctgtcttccagggtaaaaataataaggttgaatcattttattaaaaatactttttaagaaaataactatgaacatctgaatattaaagatataaaaatgcacataattcatatttcaggtggtatttgcattcagtgccttactggtattctcagaacattttaatgatttctaacatttcttaacagtcatagatatatacattttcattttttgtacttgaatattctaaataaaactgacatttactcttgacaaataaaacatatatttactaaaatgtgtttaattttcctttctgaaaactctcattttaaaaacgttcatttaattatgtatttgaattattttggagatgaggtattttatgagtattttcagacaatgaaacttattagtctgtgtcagattctgagcaatcatagagtcatctaagttgtaaataaaaccttgcatagcacaaaaaaaaaaaaaaaaa

SEQ ID NO: 2 - protein, 454 aa, Homo sapiens
MASSEVARHLLFQSHMATKTTCMSSQGSDDEQIKRENIRSLTMSGHVGFESLPDQLVNRSIQQGFCFNILCVGETGIGKSTLIDTLFNTNFEDYESSHFCPNVKLKAQTYELQESNVQLKLTIVNTVGFGDQINKEESYQPIVDYIDAQFEAYLQEELKIKRSLFTYHDSRIHVCLYFISPTGHSLKTLDLLTMKNLDSKVNIIPVIAKADTVSKTELQKFKIKLMSELVSNGVQIYQFPTDDDTIAKVNAAMNGQLPFAVVGSMDEVKVGNKMVKARQYPWGVVQVENENHCDFVKLREMLICTNMEDLREQTHTRHYELYRRCKLEEMGFTDVGPENKPVSVQETYEAKRHEFHGERQRKEEEMKQMFVQRVKEKEAILKEAERELQAKFEHLKRLHQEERMKLEEKRRLLEEEIIAFSKKKATSEIFHSQSFLATGSNLRKDKDRKNSNFL

SEQ ID NO: 3 - DNA, 4834 bp, Homo sapiens
cggcgcccgcgggctgggagccggggcccgcaggtggaagcgcacccgggaggcgggccggccggggctggagcggctcgggcgggctcttgacgctcagccagcttcgctccggcctcgggaaggcgcgcgtcccgccctgacccgccggcctctcccaccccagcagtgacgcgccgcctgggagctggagcccgcgcagcgccccgcagggcgatggacggccgaaccccgcgcccgcaggacgccccagccaggagaaaaccaaaagccaaggcaccacttcctccagctgagaccaaatatactgatgtctcttcagctgctgattctgtagaatccactgctttcatcatggaacagaaagaaaacatgatagataaagacgttgaactctcagtggtcctacctggggatattatcaaatctactactgttcatggcagtaaacctatgatggacttgttgatattcctttgtgcacagtatcacttaaatccatcaagttacacaatcgatctgttgtcagctgaacagaaccacattaaatttaagccaaacacaccaataggaatgttggaggtagagaaggtaattttaaagccaaaaatgttggataagaaaaaacctacacctataataccagagaaaactgtgagagtagtgattaattttaagaaaacacagaagaccatagtgagagtgagtccacatgcatcgcttcaagagcttgcccctattatatgtagcaaatgtgagtttgatccgttgcatacactattgttgaaagattatcaatcgcaggagcctcttgacttgacaaaatctcttaatgacctgggactaagagaattatatgcgatggatgtcaacagagagtcctgccaaatatcacaaaacctagatattatgaaggagaaagaaaataaagggtttttcagtttttttcaacgcagtaagaaaaagcgagaccaaactgcaagtgcccctgcaacccctctagtaaataagcaccgcccaacttttacaaggtccaataccatttccaaaccatatatttccaacaccctgccgtcggatgcacccaagaagaggcgggctccactgcccccgatgccagcatctcagagtgtcccccaagaccttgcacacatccaggagaggcctgcttcttgtatagtgaaatccatgagcgtggatgagacagataagagtccctgtgaagcaggaagagtgagggcaggttcactgcagctcagcagcatgtctgcagggaattcatctttgagaaggacaaagcgaaaagcaccttccccaccctccaaaatacccccgcatcaaagtgatgaaaatagtcgtgtgactgccttacagccagtagatggagttcctccagacagtgcttcagaagcaaactctcctgaggagctatccagcccagaaacctttcaccctgggctttccagtcaggagcagtgcactgcgcccaaactgatggaggaaacctctgtctttgagtgccctgggacacctgaggcagccataacatcattgacatctggaataagctctgattatagccttgaagagatagatgaaaaggaagaactgagtgaagtgcctaaagttgaagctgaaaatatttctccgaagtcacaagatattccttttgtatctactgatataataaatacactgaaaaatgatcctgactcagcccttggcaatggtagtggagagttctcacaaaactccatggaagaaaaacaagaaactaaaagcacagatggacaagaaccacacagtgtagtatatgatacaagcaatggaaagaaggtagttgacagtataagaaacttgaagtcgttgggcccaaaccaagagaatgttcaaaatgaaataattgtctatccagagaacacagaagacaatatgaaaaatggagtgaagaaaacagaaatcaatgtagaaggtgttgccaaaaataacaacattgatatggaagttgagagaccatcaaactctgaggcacatgaaactgatactgctataagttacaaggaaaaccatctagcagcttcatcagtaccagatcaaaaactgaatcaacccagtgcagaaaagacaaaagatgcagcaattcagacaaccccttcttgtaacagttttgatgggaaacaccaagatcataatttatctgactccaaagttgaagaatgtgtgcaaacttcaaataacaacatatcaactcaacactcatgcttaagttcacaagattctgtaaatacctcaagggaattcaggagtcaaggcaccctaattatacattcagaagatccgcttaccgtaaaagatccaatttgtgcacatggtaatgatgatcttttgcctcctgtagataggattgacaaaaattccactgcttcttacctaaagaattacccactttatagacaggactacaatcccaagccaaaaccttcaaatgaaattacacgagagtatatacccaaaattggcatgactacttataaaatagtgcctcccaaatccttggaaatatcgaaagactggcaatcagaaaccatagagtataaagatgatcaggacatgcatgctttagggaaaaagcacactcatgagaatgtgaaagaaactgccatccaaacagaagattctgctatttctgaaagcccagaagagccactgccaaaccttaaaccgaagcctaacctgagaacagagcatcaagtgcccagttctgtgagctcacctgatgatgccatggttagtcctctgaaacctgctcccaaaatgacaagagacactggcacagctccttttgcaccaaatttggaagaaataaacaatattttggaatcaaaatttaaatctcgggcttcaaatgcccaggccaaacccagctctttttttttgcagatgcagaagagagtatcgggtcactatgtgacatctgcagctgccaagagtgtccatgctgcccctaatcctgctccaaaagaactgacaaataaagaggcagaaagggatatgctgccttctccggagcagactctttctcccttaagtaaaatgcctcactctgttccacaaccccttgttgaaaaaactgatgatgatgtcatcggtcaggctcctgctgaagcctcccctcctcccatagctccaaaacctgtgacaattcctgctagtcaggtatccacacaaaatctgaagactttgaaaacttttggtgccccacgaccatactcaagttctggtccttcaccgtttgctcttgctgtagtgaaaaggtcacagtctttcagtaaagagcgcaccgagtcacctagtgccagtgcattggtccaacctccagccaacacagaggaagggaagactcattctgtaaataaatttgtggacatcccacagcttggtgtgtctgataaggaaaataactctgcacataatgaacagaattcccaaataccaactccaactgatggcccatcattcactgttatgagacaaagttctttaacattccaaagctctgacccagaacagatgcgacagagtttgctgactgcaatccgttcgggagaggctgctgccaaattgaaaagggttaccattccatcaaatacaatatctgtgaatggaaggtcaagactcagccattccatgtcccctgatgcccaggacggccattaaatgttaccctgccacaccactgcacttcacttccacttcagaccaacttcatactaatggaacattttggcaaatgtatattcagatgtacactaatatattatctattaaaatattagaatttgtgttgtggcttttaatgccagaagaaaagttaccagaatttataatttatagtaattttttgatcttttttttgccttaagagttgaatatgctgctttagaactttaaaacaaggtgtaaatgattttcattttttacaaatgaaaaataattcctttgtattgatttcacttaccagcacattctctacaatggtgacttagacaaaagtataagattcatagactttatatttgtatgacatacaactaggacaaacatagatatgacatttgctgcctcagtgtagcaattggaaatatttataagttatatgaaagcctgttttgggctgaaagaatgatttagaaaactagtgataccaaataagtatattcagttcaataattattttcaatgatgaatcacttagtgtgaaagacttgccttgtgtattctttatgtaattacaaatcactgtcaattttatgggaagctcatagtattttaatattttattaacatggaactcttgtttttttaatctttagaacttaaattctacaagaattttaaatattttctgtatataattatgacattgtcacacagaaattacacattttatgtgccagaagccttaaacatctttctgtgaaaatgctgatatattgtgacagttatttcacatttgatatgtagagaggaataggggttagtttatgtttatattgaaaaactttaaagactatttggaagttccagaaattctggttttaattcaagtaaaatgataaaatagtcattatatagttcagatgctaatattctaagtaataatatatatttacattgaagctaaaactgttaagcaaaacaatgcccatttgtcggcttacagctcttccggagtctagagcctgttggtgttctgtccctactttaagaatttaattgctcacttattctgaaagctttgttcaaacaagatgatattaaatttgttttcact

SEQ ID NO: 4 - protein, 1207 aa, Homo sapiens
RSASFAPASGRRASRPDPPASPTPAVTRRLGAGARAAPRRAMDGRTPRPQDAPARRKPKAKAPLPPAETKYTDVSSAADSVESTAFIMEQKENMIDKDVELSVVLPGDIIKSTTVHGSKPMMDLLIFLCAQYHLNPSSYTIDLLSAEQNHIKFKPNTPIGMLEVEKVILKPKMLDKKKPTPIIPEKTVRVVINFKKTQKTIVRVSPHASLQELAPIICSKCEFDPLHTLLLKDYQSQEPLDLTKSLNDLGLRELYAMDVNRESCQISQNLDIMKEKENKGFFSFFQRSKKKRDQTASAPATPLVNKHRPTFTRSNTISKPYISNTLPSDAPKKRRAPLPPMPASQSVPQDLAHIQERPASCIVKSMSVDETDKSPCEAGRVRAGSLQLSSMSAGNSSLRRTKRKAPSPPSKIPPHQSDENSRVTALQPVDGVPPDSASEANSPEELSSPETFHPGLSSQEQCTAPKLMEETSVFECPGTPEAAITSLTSGISSDYSLEEIDEKEELSEVPKVEAENISPKSQDIPFVSTDIINTLKNDPDSALGNGSGEFSQNSMEEKQETKSTDGQEPHSVVYDTSNGKKVVDSIRNLKSLGPNQENVQNEIIVYPENTEDNMKNGVKKTEINVEGVAKNNNIDMEVERPSNSEAHETDTAISYKENHLAASSVPDQKLNQPSAEKTKDAAIQTTPSCNSFDGKHQDHNLSDSKVEECVQTSNNNISTQHSCLSSQDSVNTSREFRSQGTLIIHSEDPLTVKDPICAHGNDDLLPPVDRIDKNSTASYLKNYPLYRQDYNPKPKPSNEITREYIPKIGMTTYKIVPPKSLEISKDWQSETIEYKDDQDMHALGKKHTHENVKETAIQTEDSAISESPEEPLPNLKPKPNLRTEHQVPSSVSSPDDAMVSPLKPAPKMTRDTGTAPFAPNLEEINNILESKFKSRASNAQAKPSSFFLQMQKRVSGHYVTSAAAKSVHAAPNPAPKELTNKEAERDMLPSPEQTLSPLSKMPHSVPQPLVEKTDDDVIGQAPAEASPPPIAPKPVTIPASQVSTQNLKTLKTFGAPRPYSSSGPSPFALAVVKRSQSFSKERTESPSASALVQPPANTEEGKTHSVNKFVDIPQLGVSDKENNSAHNEQNSQIPTPTDGPSFTVMRQSSLTFQSSDPEQMRQSLLTAIRSGEAAAKLKRVTIPSNTISVNGRSRLSHSMSPDAQDGH

SEQ ID NO: 5 - DNA, 2243 bp, Homo sapiens
atgcgggcgcgtggccccgcccccaaacaaggccacgcggcgtgcgctcacccgcctctcggggacagtaccaccagccccggcctgaggccccgacccctcagcgccacacgcacaagcgcgttcgcacgattggtgcaggcggacgcgcgaggtcccgcgcctgcgcacacccccgaggctggcacgcacaccggtctcctaggggactggcgcctggccctgcttttcatcctctcaggagatcacgtgtggacactgaggcccttcctcgagctctttaaccaaacccaacctccccaattcgccgccttccccgctcccacaaccacacgttcccgtgtgagggcttattgcctccagccagccagcgtccttcgcccccacattcacgaggtttggagccggcctcacccgcggaggcggacaccgcccacccgcgcgtgcgcactacgcagtcatcctgcgacctccagaatccaccggggtgcagcgaggctgtggaaagtcccacccagaacgtgaggtgaagaaggcttgggctgcgctggttctgctctgtccgggtcggagatgaactgacccgggagagcagcggagggatgtttcttttggccaacagtggggactcacctgcacgttttgcgaagaggcccacagtcctttgcgtggcgctcggactacatttcccaacggcccctgcacgccctgggggctgttccatgcggtgttgcgcctgcgtagccggcgggctggcagtgagactgactgcgtcggggttgagactgggtggatgaggctcaccccggcggggagaagggacgaggaggggcggacagcggaaggtccgggagtgtccgccataaagtcgtttgaggtgaccgttgcgtaattgtgagtctgtgagagaagatgtgaagtatggcctcgtcccggtcatctgggcgtgcgggtcccgggttttgatcgcgcgtttgtgtagttttaacttctagtcatggcgaatgatcgcaggagagcacagactggaccctgctacgatctctcttggagtggatcagactgatgatcaccaacaaccaactcattcccggataaggaagaagagagtgtcacctacttcagtgtggtttcaaccctacttctgcatcttaaagacactgtatggtttcagcagtagtgcccctgttcattagtccccctgatgttttcattcctcatctcatctttttcttagcagcattcaatgaatccttcattctagaaacactctatatctttggttttcatgagaccattctcaccttgttttgtcctgtgacttttttgaaaaaaacaaaaacaaaaaaccctttttttctttttaaattctggtaaaaaacacaatgaaaatttgctatcttaaccatgttgaaatgtgcagttagtaaagtacattcacattgtggtgcaagccatcactaccatccatcactagaacccttttcatcttgcagatctgaaactctacccattaaacgacttcccatcttcccatccccacagctcctagcaaccaacattctactttctctatcagtttgactactctaggtacctcatatgagtagaatcatacagcatttatccttctctgcctggcttatttcacttgtataatgtcctcaaggttcattcatgttgtagcatgcatcagaacttcctccccttttaaaggctggataatatttcatggtatgtttagatcacattctgtttatccattcatccatcagtgaacacttgtgctccttccaactttgggctgttgggtgtcctgccactgttgctcctagtgctcaatctcgtttattccctcctaatcaagtgtacaacgttggacactgtgcaggatgatgccacttcatcttggatgctaatctgccatgttgacttctgattaaccccaggcccaggaatgcctcaagatttctactttacttactgttgcttgtgtaagccaagacaaccttgatgttatcataaacatgtacttacctaagtcctgtcctttggcaaattatgggctatgagacacagcattcttgcctttccctgaggggtcaatttcagcgatcctacacattccttctgaagcacttatgctctttctatatggtatgtaagctctcggtctggggagtaacagtgcagagatctacctgtcttgttgccacatgtttctaaactttccaataaatcac

SEQ ID NO: 6 - protein, 174 aa, Homo sapiens
MRARGPAPKQGHAACAHPPLGDSTTSPGLRPRPLSATRTSAFARLVQADARGPAPAHTPEAGTHTGLLGDWRLALLFILSGDHVWTLRPFLELFNQTQPPQFAAFPAPTTTRSRVRAYCLQPASVLRPHIHEVWSRPHPRRRTPPTRACALRSHPATSRIHRGAARLWKVPPRT

SEQ ID NO: 7 - DNA, 2958 bp, Homo sapiens
caagcaggttagagtacaaaacatgttctccctgcagtctcacgaactgtgaacaaaaactgaagtgaaaactcatagtgcataacttgtcaatactcctgtgatcgtataaccatcagcaagaaaacaaatttgattgagcccccatccagtcctctttgcgtggaatcagacctcttttgcagtggaaaggagcagtgctgcagctctgatggttcaactctgccaaaagatggatctttaatgattagcactacacactgaccaactcagaagaaggagccacaccacctgtgactccagctctgacttctgctctggaccagtgtttccataacagggacttcaaaatcactgtgatttgaagcctttttgaacatgaagatgttactcctgctgcattgccttggggtgtttctgtcctgttctggacacatccaggatgagcacccccaatatcacagccctccggatgtggtgattcctgtgaggataactggcaccaccagaggcatgacacctccaggctggctctcctatatcctgccctttggaggccagaaacacattatccacataaaggtcaagaagcttttgttttccaaacacctccctgtgttcacctacacagaccagggtgctatccttgaggaccagccatttgtccagaataactgctactatcatggttatgtggaaggggacccagaatccctggtttccctcagtacctgttttgggggttttcaaggaatattacagataaatgactttgcttatgaaatcaagcccctagcattttctaccacgtttgaacatctggtatacaagatggacagtgaggagaaacaattttcaaccatgagatccggatttatgcaaaatgaaataacatgccgaatggaatttgaagaaattgataattccactcagaagcaaagttcttatgtgggctggtggatctattttaggattgttgaaattgtagtcgtcattgataattatctgtacattcgttatgaaaggaacgactcaaagttgctggaggatctatatgttattgttaatatagtggattccattttggatgtcattggtgttaaggtgttattatttggtttggagatctggaccaataaaaacctcattgtagtagatgatgtaaggaaatctgtgcacctgtattgcaagtggaagtcggagaacattacgccccggatgcaacatgacacctcacatcttttcacaactctaggattaagagggttaagtggcataggagcttttagaggaatgtgtacaccacaccgtagttgtgcaattgttactttcatgaacaaaactttgggcactttttcaattgcagtggctcatcatctaggtcataatttgggcatgaaccatgatgaggatacatgtcgttgttcacaacctagatgcataatgcatgaaggcaacccaccaataactaaatttagcaattgtagttatggtgatttttgggaatatactgtagagaggacaaagtgtttgcttgaaacagtacacacaaaggacatctttaatgtgaagcgctgtgggaatggtgttgttgaagaaggagaagagtgtgactgtggacctttaaagcattgtgcaaaagatccctgctgtctgtcaaattgcactctgactgatggttctacttgtgcttttgggctttgttgcaaagactgcaagttcctaccatcagggaaagtgtgtagaaaggaggtcaatgaatgtgatcttccagagtggtgcaatggtacttcccataagtgcccagatgacttttatgtggaagatggaattccctgtaaggagaggggctactgctatgaaaagagctgtcatgaccgcaatgaacagtgtaggaggatttttggtgcaggcgcaaatactgcaagtgagacttgctacaaagaattgaacaccttaggtgaccgtgttggtcactgtggtatcaaaaatgctacatatataaagtgtaatatctcagatgtccagtgtggaagaattcagtgtgagaatgtgacagaaattcccaatatgagtgatcatactactgtgcattgggctcgcttcaatgacataatgtgctggagtactgattaccatttggggatgaagggacctgatattggtgaagtgaaagatggaacagagtgtgggatagatcatatatgcatccacaggcactgtgtccatataaccatcttgaatagtaattgctcacctgcattttgtaacaagaggggcatctgcaacaataaacatcactgccattgcaattatctgtgggaccctcccaactgcctgataaaaggctatggaggtagtgttgacagtggcccaccccctaagagaaagaagaaaaagaagttctgttatctgtgtatattgttgcttattgttttgtttattttattatgttgtctttatcgactttgtaaaaaaagtaaaccaataaaaaagcagcaagatgttcaaactccatctgcaaaagaagaggaaaaaattcagcgtcgacctcatgagttacctccccagagtcaaccttgggtgatgccttcccagagtcaacctcatgtgacaccttaccagagtcatcctcaggtgatgccttcccagagtcaacctcctgtgacaccctcccagagtcaacctcgggtgatgccttctcagagtcaacctcctgtgatgccttcccagagtcatcctcagttgacgccttcccagagtcaacctcctgtgacaccctcccagaggcaacctcagttgatgccttcccagagtcaacctcctgtgacgccctcctagagccagcctcagttgatgccttcccagagtcaacctcctgtgacgccctcccagagccaacctcgggtgacaccctcccagagtcaacctcatgtgacaccttaccggagtaaaagt

SEQ ID NO: 8 - protein, 820 aa, Homo sapiens
MKMLLLLHCLGVFLSCSGHIQDEHPQYHSPPDVVIPVRITGTTRGMTPPGWLSYILPFGGQKHIIHIKVKKLLFSKHLPVFTYTDQGAILEDQPFVQNNCYYHGYVEGDPESLVSLSTCFGGFQGILQINDFAYEIKPLAFSTTFEHLVYKMDSEEKQFSTMRSGFMQNEITCRMEFEEIDNSTQKQSSYVGWWIYFRIVEIVVVIDNYLYIRYERNDSKLLEDLYVIVNIVDSILDVIGVKVLLFGLEIWTNKNLIVVDDVRKSVHLYCKWKSENITPRMQHDTSHLFTTLGLRGLSGIGAFRGMCTPHRSCAIVTFMNKTLGTFSIAVAHHLGHNLGMNHDEDTCRCSQPRCIMHEGNPPITKFSNCSYGDFWEYTVERTKCLLETVHTKDIFNVKRCGNGVVEEGEECDCGPLKHCAKDPCCLSNCTLTDGSTCAFGLCCKDCKFLPSGKVCRKEVNECDLPEWCNGTSHKCPDDFYVEDGIPCKERGYCYEKSCHDRNEQCRRIFGAGANTASETCYKELNTLGDRVGHCGIKNATYIKCNISDVQCGRIQCENVTEIPNMSDHTTVHWARFNDIMCWSTDYHLGMKGPDIGEVKDGTECGIDHICIHRHCVHITILNSNCSPAFCNKRGICNNKHHCHCNYLWDPPNCLIKGYGGSVDSGPPPKRKKKKKFCYLCILLLIVLFILLCCLYRLCKKSKPIKKQQDVQTPSAKEEEKIQRRPHELPPQSQPWVMPSQSQPHVTPYQSHPQVMPSQSQPPVTPSQSQPRVMPSQSQPPVMPSQSHPQLTPSQSQPPVTPSQRQPQLMPSQSQPPVTPS

(19) United States
(12) Patent Application Publication
(10) Pub. No.: US 2008/0280297 A1
(43) Pub. Date: Nov. 13, 2008

(54) Compositions and Methods for Differential Diagnosis of Chronic Lymphocytic Leukemia

(75) Inventor: Riccardo Dalla-Favera, New York, NY (US)

Correspondence Address: WilmerHale/Columbia University, 399 PARK AVENUE, NEW YORK, NY, 10022, US

(73) Assignee: THE TRUSTEES OF COLUMBIA UNIVERSITY IN THE CITY OF, New York, NY (US)

(21) Appl. No.: 11/995,719

(22) PCT Filed: Jul. 17, 2006
(86) PCT No.: PCT/US2006/027641

§ 371 Date: Jul. 24, 2008

Related U.S. Application Data
(60) Provisional application No. 60/699,694, filed on Jul. 15, 2005.

(51) Int. Cl. G01N 33/574; C12Q 1/68; C07K 16/00

(52) U.S. Cl. 435/6; 530/387.1; 435/7.23

(57) ABSTRACT

The invention provides compositions and methods for determining a prognosis of a B cell chronic lymphocytic leukemia (CLL) in a subject based on the level of expression of at least one marker gene. Marker genes provided by the invention are SEPTlO, KIAA0799, Hs.23133, and ADAM29. The marker genes can be used to differentially diagnose CLL in a subject based on relative gene expression levels in the subject compared to reference gene expression levels established from a clinically characterized population of patients. The invention also provides diagnostic reagents and compositions and kits based on the marker genes.

This application claims priority to U.S. Provisional Application No. 60/699,694 filed on Jul. 15, 2005, which is hereby incorporated by reference in its entirety.

The invention disclosed herein was made with U.S. Government support under NIH Grant No. 072699 from the National Cancer Institute. Accordingly, the U.S Government may have certain rights in this invention.

### 1. INTRODUCTION

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described herein.

The present invention relates to methods for determining the prognosis of chronic lymphocytic leukemia in a subject based on the levels of expression of a set of marker genes. The invention also encompasses reagents for use in the methods, and test kits.

### 2. BACKGROUND OF THE INVENTION

B cell Chronic Lymphocytic Leukemia (CLL) occurs almost exclusively in adults with a median age at diagnosis of 65 to 68 years old. It comprises approximately 10% of all adult hematologic malignancies, but 40% of leukemias in individuals over 65 years of age. In the United States, approximately 8,000 new cases are diagnosed each year, with a worldwide incidence of 3-4 per 100,000 per year. Epidemiologic studies have shown the incidence to be higher in North American white and black populations, Europe, and Australia, than in India, China, and Japan. However for all populations, CLL is more prevalent in males than females (2:1).

There is a general belief that CLL is an indolent disease associated with a prolonged (up to 10-20 years) clinical course, and that the eventual cause of death may be unrelated to CLL. This observation, however, is true for less than 30 percent of all CLL cases. Some patients die rapidly, within two to three years from diagnosis, from complications or causes directly related to CLL. Many patients live for 5 to 10 years with an initial course that is relatively benign but almost always followed by a terminal phase lasting one to two years during which there is considerable morbidity, both from the disease itself and from complications of therapy. In a variable percent of patients with CLL, and usually as a terminal event, CLL transforms into another lymphoproliferative disorder. The following are the most commonly reported transformations: prolymphocytic leukemia, diffuse large B-cell lymphoma (Richter's transformation), Hodgkin's disease, and multiple myeloma.

During the initial asymptomatic phase, patients are able to maintain their usual lifestyles, but during the terminal phase the performance status is poor, with recurring need for hospitalization. The most frequent causes of death are severe systemic infection (especially pneumonia and septicemia), bleeding, and inanition with cachexia.

CLL arises through clonal expansion of B lymphocytes. Conventional karyotypic analyses of the leukemic cells proved to be difficult due to the paucity of dividing leukemic cells. Few studies have reported chromosomal abnormalities associated with CLL, with particular note of lack of reciprocal balanced translocations, presence of specific deletions, and correlation between patients exhibiting a normal karyotype or 13q- with a better survival, and those exhibiting a complex karyotype or trisomy 12 with a poorer survival (Juliusson G et al., Prognostic subgroups in B-cell chronic lymphocytic leukemia defined by specific chromosomal abnormalities. N Engl J Med 323:720-724, 1990). Application of interphase fluorescence in situ hybridization (FISH) analysis to the study of chromosomal abnormalities in CLL using comprehensive panels of probes has been highly informative in revealing abnormalities that previously went unrecognized in CLL and redefining the frequencies of those already known. These studies indicated the prognostic significance in multivariate analysis of 17p- and 11q-associated with shorter survival. In the former case, the target gene is thought to be TP53, since a high proportion of CLL patients (26%) exhibit abnormal p53 function (Dohner H et al., Genomic aberrations and survival in chronic lymphocytic leukemia. N Engl J Med 343:1910-1916, 2000; Lin K et al., Relationship between p53 dysfunction, CD38 expression, and IgV(H) mutation in chronic lymphocytic leukemia. Blood 100:1404-1409, 2002).

It was originally believed that the cell of origin of CLL was a naive B lymphocyte that had not undergone germinal center (GC) antigen exposure nor associated somatic hypermutation of their immunoglobulin genes. The observation that approximately half of CLL patients exhibit somatic mutations within the variable (V) region of the immunoglobulin heavy chain gene (IGH), a phenomenon occurring in normal B cells upon T cell-dependent GC reaction and in malignant B cells derived from GC or post-GC B cells, led to the hypothesis that CLL may arise from either a B cell that had transited through the GC (mutated IgV), or a GC-independent cell (non-mutated IgV). Comparison of expression profiles of CLL B cells displaying IgV mutation versus unmutated revealed a restricted set of expression differences between the two subtypes, though fewer than expected if the two subtypes were to be derived from different B cell subpopulations (Klein et al., Gene expression profiling of B cell chronic lymphocytic leukemia reveals a homogeneous phenotype related to memory B cells. J Exp Med 194:1625-1638, 2001; Rosenwald A et al., Relation of gene expression phenotype to immunoglobulin mutation genotype in B cell chronic lymphocytic leukemia. J Exp Med 194:1639-1647, 2001).

During the past 30 years, a shift has occurred in the pattern of CLL diagnosis. In the past, patients presented with lymphadenopathy, systemic symptoms such as tiredness, night sweats, and weight loss, or the symptoms of anemia or infection. At the present time, CLL is often detected in asymptomatic patients with an elevated lymphocyte count in a routine full blood count. Definitive diagnosis is based on a lymphocytosis and characteristic lymphocyte morphology and immunophenotype. Two major staging systems for the disease exist: Rai and Binet (Shanafelt T D, et al., Prognosis at diagnosis: integrating molecular biologic insights into clinical practice for patients with CLL. Blood 103:1202-1210, 2004; and British Society of Hematology. Guidelines on the diagnosis and management of chronic lymphocytic leukemia. Brit J Haematol 125: 294-317, 2004). The former was based on the presence of lymphadenopathy, organomegaly (spleen and liver), and cytopenias, with five stages. The Binet system places patients into three stages and was based on similar disease burden measures as Rai with greater prognostic significance placed on those features which in retrospective studies correlated with survival.

These staging systems have been useful in stratifying patients for clinical research studies, and have guided the care and treatment approaches of these patients. Those with early stage CLL often will not be treated, utilizing a “watchful waiting” approach for this slowly progressive form of the disease. The staging systems however, do not permit the identification of a significant proportion of patients with early stage disease that unexpectedly become active and refractory to treatment. Patients with late stage CLL are treated with chemotherapy in combination with monoclonal antibodies as for aggressive non-Hodgkin's lymphomas, with refractory/relapsed patients targeted for autologous and allogeneic stem cell transplantation (allo-SCT). In the case of allo-SCT, some graft-versus-leukemia activity has been evidenced. Again, the staging systems do not identify patients with stable versus aggressive late stage disease. Thus, for a disease entity that presents predominantly in an aging population, accurate prognostication for treatment options is highly desirable.

An important clinical challenge in CLL is the identification of patients who will exhibit a slow stable/progressive course versus those with refractory or aggressive disease requiring aggressive treatment regimens. Prognostication of CLL had predominantly involved risk stratification by stage, and variously lymphocyte doubling time, serum α-2 microglobulin levels, and interphase FISH analysis for specific chromosomal abnormalities, until the observation that IGHV mutational status is of prognostic significance (Hamblin T J et al., Unmutated Ig V(H) genes are associated with a more aggressive form of chronic lymphocytic leukemia. Blood 94:1848-1854, 1999; Damle R N et al., Ig V gene mutation status and CD38 expression as novel prognostic indicators in chronic lymphocytic leukemia. Blood 94:1840-1847, 1999). It has now been clearly established that CLL patients with B cells that exhibit an IGHV gene that differs from germline by ≧2% in the V region have a significantly better outcome than patients whose B cells exhibit little or no evidence of IGHV mutation. The underlying biologic basis for this association remains unknown. Unfortunately due to the labor intensity and inherent technical difficulties of the performance of the PCR and sequencing based assay for IGHV mutation analysis, this assay is rarely performed outside of a research-linked clinical setting. To this end, identification of surrogate markers for IGHV mutational status that are easily performed has become a focus of research efforts. The first reported surrogate marker evaluated by flow cytometry was CD38 whose expression was elevated in those CLL not exhibiting mutation (U.S. Pat. No. 6,506,551). Subsequent reports did not confirm the correlation (Hamblin T J et al., CD38 expression and immunoglobulin variable region mutations are independent prognostic variables in chronic lymphocytic leukemia, but CD38 expression may vary during the course of the disease. Blood 99:1023-1029, 2002). Examination of the expression profiles between CLL with and without IGHV mutation lead to the recent evaluation of another such identified surrogate marker: ZAP-70. Two recent reports using flow cytometry of whole blood or isolated mononuclear cells have detailed a correlation between expression in ≧20% of leukemic cells of ZAP-70 with lack of IGHV mutation and poorer outcome (Crespo M et al., ZAP-70 expression as a surrogate for immunoglobulin-variable-region mutations in chronic lymphocytic leukemia. New Engl J Med 348:1764-1775, 2003; Orchard J A et al., ZAP-70 expression and prognosis in chronic lymphocytic leukemia. Lancet 363:105-111, 2004). The correlation has yet to be validated or confirmed by other researchers, and it should be noted that the correlation between ZAP-70 expression and IGHV mutational status was discordant in approximately 10% of cases.

With the well-documented increase of an aging population within the United States, it is expected that the number of newly diagnosed cases of CLL will increase accordingly. Clearly, the challenge amongst these patients and indeed all CLL patients at diagnosis is to determine which of these patients will have an indolent versus an aggressive clinical course. At the present time, risk stratification is still largely based on clinical criteria, as few biologic markers have proven robust. IGHV mutational status would appear at the present time to be the most robust molecular marker of clinical course, though as indicated above, this assay is not easily performed in a routine setting. Recent studies have identified two surrogate markers for IGHV mutational status, one of which (CD38) has proven to be unreliable, with the second (ZAP-70) pending further evaluation. Therefore, there is an urgent need for reliable and convenient methods to determine the prognosis of CLL in these patients.

### 3. SUMMARY OF THE INVENTION

The invention relates to methods for determining a prognosis for B cell chronic lymphocytic leukemia (CLL) in a human subject. The invention also encompasses the marker polynucleotides and polypeptides used for the prognosis and/or diagnosis of CLL, diagnostic reagents, diagnostic compositions, and related diagnostic kits.

The present invention is based in part on the discovery that four marker genes, namely SEPT10, KIAA0977, Hs.23133 and ADAM29, are of particular utility in determining the prognosis of CLL. The inventors also recognize that the markers may be useful in selecting an appropriate therapeutic regimen, and/or to predict the ability of an individual to respond to a particular agent. Accordingly, the invention provides assays for predicting the benefit of a treatment regimen for a subject with CLL. Also encompassed are assays for determining the associations between expression of the markers with a clinical condition or treatment outcome. Non-limiting examples of additional genes that can be used as markers within the context of this invention include AICL (activation-induced C-type lectin), septin II-like cell division protein, dystrophin DMD, gravin, fibroblast muscle-type tropomyosin, photolyase, kallikrein, lipoprotein lipase, BCL7A, calcireticulin, KCNG1, WSB-2, V4-31 Ig variable region, dipeptidyl peptidase IV, CD30, LDOC1, phorbolin-like protein MDSO19, FGL2 (fibrinogen-like protein 2), and MEGT1 (Klein et al., J Exp Med 194:1625-1638 (2001)).

The present invention provides isolated marker polynucleotides or variants thereof, which can be used, for example, as hybridization probes or primers (“marker probes” or “marker primers”) to detect or amplify nucleic acids encoding a marker polypeptide. The present invention also provides “marker antibodies” that immunospecifically binds to the respective marker proteins or polypeptides. Compositions comprising labeled marker polynucleotides, or labeled marker antibodies are also encompassed by the invention.

The invention further encompasses use of the marker polynucleotides and/or marker proteins in combination with other means of providing a prognosis for CLL, such as uses of other genes (e.g., ZAP70 and/or CD38), determination of the mutational status of immunoglobulin genes, cytogenetics observations, and clinical observations.

In one embodiment, the invention provides a method for determining a prognosis for B cell chronic lymphocytic leukemia in a subject, said method comprises the steps of obtaining test cells from a subject in need of prognostic information, determining the level of expression of at least one marker gene in the test cells, wherein said at least one marker gene is SEPT10, KIAA0799, Hs.23133, or ADAM29; and determining the prognosis based on the level of expression of at least one of the marker gene in the test cells. According to the invention, a high level of expression of SEPT10 relative to a reference SEPT10 level indicates a prognosis of aggressive CLL; a high level of expression of Hs.23133 relative to a reference Hs.23133 level indicates a prognosis of aggressive CLL; a low level of expression of KIAA0799 relative to a reference KIAA0799 level indicates a prognosis of indolent CLL; and a low level of expression of ADAM29 relative to a reference ADAM29 level indicates a prognosis of indolent CLL. The reference SEPT10 level, reference KIAA0799 level, reference Hs.23133 level, and/or reference ADAM29 level can be established from cells from characterized cell lines, or cells from a clinically-characterized population of patients, such as but not limited to, patients that have the aggressive form of the disease, patients that have the indolent form of the disease, or patients displaying mutations in the genes encoding immunoglobulin heavy chain variable regions, patients with no mutation in these genes.

The test cells obtained from the subject, depending on the marker and the assay method used may comprise chronic lymphocytic leukemia cells, CD5+/CD19+/CD23+ cells, CD5+/CD19+ cells, CD19+/CD23+ cells, CD5+/CD23+ cells, and/or B cells. In other embodiments, the test cells are peripheral mononuclear blood cells, or whole blood cells.

Various assay methods can be used to determine the level of marker gene expression in the test cells. In one embodiment, the level of expression is determined by measuring the amount of marker polypeptide. Other embodiments of the invention encompass the steps of contacting a marker antibody with a sample of test cells under conditions that allow the antibody to bind to marker polypeptides on the surface of or inside the test cells; and detecting or measuring binding of the marker antibody to the marker polypeptides. The term “contacting” is used herein interchangeably with the following: introducing into, combined with, added to, mixed with, passed over, incubated with, injected into, flowed over. In another embodiment, the level of expression is determined by measuring the ratio of test cells expressing said the marker in a batch of test cells relative to the total number of test cells. The ratio of positive test cells (i.e., cells expressing the marker) relative to the total number of test cells can be measured by flow cytometry, and a prognosis is determined if the ratio is above or below a cut-off reference ratio. Such a reference ratio can be determined using test cells obtained from clinically-characterized patients and/or cell lines of a known genotype/phenotype.

In another embodiment, the level of expression of a marker gene is determined by measuring the amount of marker messenger RNA, for example, by DNA-DNA hybridization, RNA-DNA hybridization, reverse trans cription-polymerase chain reaction (PCR), or real time quantitative PCR; and comparing the results to a reference based on samples from clinically-characterized patients and/or cell lines of a known genotype/phenotype.

The invention also provides compositions comprising marker polynucleotides, maker polypeptides, or marker antibodies. In one embodiment, the Hs.23133 proteins, polypeptides, and antibodies are included. The invention further provides diagnostic reagents for use in the methods of the invention, such as but not limited to reagents for flow cytometry and/or immunoassays that comprise a fluorochrome-labeled anti-SEPT10 antibody, a fluorochrome-labeled anti-KIAA0799 antibody, a fluorochrome-labeled anti-Hs.23133 antibody, or a fluorochrome-labeled anti-ADAM29 antibody. Other non-limiting examples of diagnostic reagents comprise a) at least one of anti-CD5 antibody, anti-CD19 antibody, and/or anti-CD23 antibody; and b) at least one of anti-SEPT10 antibody, anti-KIAA0799 antibody, anti-Hs.23133 antibody, and/or anti-ADAM29 antibody.

In another embodiment, the invention provides diagnostic compositions comprising compositions of the invention and materials from test subjects. Such diagnostic compositions are made when the methods of the invention are practiced with compositions, diagnostic reagents, or components of diagnostic kits of the invention. Typically, during an assay, test cells or materials from test cells are contacted with a diagnostic reagent of the invention. For example, a diagnostic composition may comprise (a) at least one of anti-SEPT10 antibody, anti-KIAA0799 antibody, anti-Hs.23133 antibody, and/or anti-ADAM29 antibody; and b) test cells comprising chronic lymphocytic leukemia cells, CD5+/CD19+/CD23+ cells, CD5+/CD19+ cells, CD19+/CD23+ cells, CD5+/CD23+ cells, and/or B cells, said test cells being obtained from a human in need of a prognosis of chronic lymphocytic leukemia. In another example, a diagnostic composition may comprise (a) at least one of SEPT10 polynucleotides, KIAA0799 polynucleotides, Hs.23133 polynucleotides, and/or anti-ADAM29 polynucleotides; and (b) nucleic acids obtained from test cells of a human in need of a prognosis of chronic lymphocytic leukemia.

The invention also provides a test kit comprising a diagnostic reagent comprising at least one of anti-SEPT10 antibody, anti-KIAA0799 antibody, anti-Hs.23133 antibody, and/or anti-ADAM29 antibody; and instructions for using the diagnostic reagent(s) in providing a prognosis of chronic lymphocytic leukemia.

### 4. BRIEF DESCRIPTION OF THE DRAWINGS

FIG. 1. Expression of five putative candidate transcripts that discriminates mutated versus unmutated CLL. ZAP70 was previously identified as a candidate, and the present study showed four new markers (SEPT10, KIAA0977, Hs.23133, and ADAM29). In all cases, the level of expression represents the average of eight mutated CLL cases (solid bars) and eight unmutated CLL cases (shaded bars). The data were generated by hybridization to the human genome oligonucleotide microarray-U133Plus2.0 by Affymetrix. Error bars show the standard deviation.

FIGS. 2A-2D. Levels of expression of SEPT10 and ZAP70, KIAA0977 and ZAP70, Hs.23133 and ZAP70, and ADAM29 and ZAP70 in unmutated and mutated CLL cases. The data were generated by hybridization to the human genome oligonucleotide microarray-U133Plus2.0 by Affymetrix.

FIG. 3. Expression of KIAA0977, SEPT10, and ZAP70 in purified lymphoid cells. The values given represent the average for five each of naïve B cells, centroblasts, centrocytes, memory B cells, T cells, and eight each of mutated and unmutated CLL. The data were generated by hybridization to the human genome oligonucleotide microarray-U133Plus2.0 by Affymetrix.

FIG. 4. Homo sapiens septin 10 (SEPT10), transcript variant 1, mRNA (GenBank accession number NM_{—}144710) (SEQ ID NO:1).

FIG. 5. The SEPT10 amino acid sequence encoded by SEQ ID NO:2 (GenBank accession number NP_{—}653311) (SEQ ID NO:2).

FIGS. 6A-6B. Homo sapiens mRNA for KIAA0977 (GenBank accession number AB023194) (SEQ ID NO:3).

FIG. 7. The KIAA0977 amino acid sequence encoded by SEQ ID NO:3 (GenBank accession number AB023194) (SEQ ID NO:4).

FIG. 8. Predicted mRNA sequence encoding Homo sapiens hypothetical protein MGC9913 (MGC9913) (GenBank accession number XM_{—}378178.2) (SEQ ID NO:5).

FIG. 9. The hypothetical amino acid sequence encoded by SEQ ID NO:5 (GenBank accession number XM_{—}378178) (SEQ ID NO:6).

FIG. 10. Homo sapiens disintegrin and metalloproteinase domain 29 (ADAM29) mRNA (GenBank accession number AF134708) (SEQ ID NO:7).

FIG. 11. The ADAM29 amino acid sequence encoded by SEQ ID NO:7 (GenBank accession number AF134708) (SEQ ID NO: 8).

### 5. DETAILED DESCRIPTION OF THE INVENTION

The invention relates to methods for determining a prognosis for B cell chronic lymphocytic leukemia (CLL) in a human subject. Also encompassed by the invention are protocols and diagnostic compositions designed for the determination of a prognosis of B-cell CLL. The present invention is based, in part, on the discovery that four marker genes, namely SEPT10, KIAA0977, Hs.23133 and ADAM29, are of particular utility in predicting the course of CLL in a patient, thereby providing useful information to the clinician in selecting the optimal modality of treatment, and to the patient in preparation for a change in his/her condition. This is especially valuable when CLL patients are being diagnosed at an increasingly early age, and more options in treatment modalities are becoming available.

A number of genes had been studied by hybridization assays for a possible association of their expression with the progression of CLL and IgV mutation status, but many such candidate genes are not suitable for clinical use as a prognostic marker. The inventors tested and selected the markers of the invention on the basis of a robust and significantly discernible differential in the levels of expression between the different disease phenotypes and/or IgV mutation status, and a low to negligible background level of expression in T cells.

CLL is characterized by the monoclonal expansion of B lymphocytes in the peripheral blood, bone marrow, and lymphoid organs, and by an indolent course which ultimately becomes aggressive and invariably lethal. Onset of CLL is usually insidious, and is often initially diagnosed from incidental blood tests or during evaluation of asymptomatic lymphadenopathy. In an asymptomatic patient, CLL may be diagnosed from abnormal blood counts. The symptomatic patient usually has nonspecific complaints of fatigue, anorexia, weight loss, dyspnea on exertion, or a sense of abdominal fullness (from an enlarging spleen or palpable nodes). Initial findings include generalized lymphadenopathy and minimal-to-moderate hepatomegaly and splenomegaly. With progressive disease, there may be pallor due to anemia. The hallmark of CLL is sustained, absolute lymphocytosis (>10,000/μL) and increased lymphocytes (>30%) in the bone marrow. The methods of the invention are applicable to symptomatic and asymptomatic CLL patients, or a subject diagnosed with CLL.

Although CLL is progressive, some patients may be asymptomatic for years. So far, there are no known treatments that will definitively increase the life expectancy of persons diagnosed with CLL, thus it is often difficult to decide when to begin aggressive treatment with the possibility that such treatment will prematurely diminish a patient's quality of life. Therefore, aggressive therapy such as radiation therapy, chemotherapy, transplants and immunotherapy is not applied until active progression or symptoms occur. However, these patients may have developed secondary malignancies, or are simply too weak to face the demands and side effects of aggressive treatment. Accordingly, the methods of the invention provide a prediction of whether the course of CLL in a subject will be indolent and slowly progressive, or aggressive which will become active and refractory to treatment. Clinical staging is useful for prognosis and treatment, and can be combined with the methods of the invention. Two common approaches to staging are the Rai system, which is primarily based on hematologic changes, and the Binet system, based on extent of disease.

The present inventors discovered a striking correlation between the expression levels of the markers and the presence of somatic mutations within the variable region of the immunoglobulin heavy chain gene, i.e., the IgV mutation status. Accordingly, the markers of the invention can also acts as a surrogate for the IgV mutation status of a subject. The methods of the invention are applicable to patients of which the IgV mutation status is not determined.

Furthermore, because the knowledge of pathogenesis of CLL is limited and the progression of CLL is heterogeneous, it is difficult to distinguish patients who are responding to a therapeutic modality being administered from patients who would have never progressed to a more advanced stage of the disease regardless of treatment. Accordingly, the methods of the invention also afford clinicians a more reliable method for evaluating treatment options, as well as optimizing the treatment regimen. The methods of the invention are thus applicable to CLL patients that are not yet under treatment, or that are receiving one or more treatment modalities.

The present invention generally discloses methods for determining the prognosis of a subject with B cell chronic lymphocytic leukemia, comprising determining the level of expression of a marker gene in the test cells of the subject, and comparing the level of expression of the marker gene in the subject's test cells to a reference level of expression of the marker gene in standard test cells. In one embodiment, the test cells used in the methods comprise CLL cells.

As used herein, the term CLL cells (B-CLL) refers to cells characterized by the expression of the cell surface markers CD5, CD23, CD19, and low levels of surface IgM and surface IgD, a pattern not shared by any known B cell subpopulation. In comparison to lymphomas, CLL cells do not express or express weakly CD22, CD79b, CD10, and FMC7.

As used herein, the phrase “marker gene expression” refers to transcription of a marker gene which produces marker pre-mRNA, marker mRNA, and/or translation of marker mRNA to produce marker polypeptide or marker protein. “Differential expression,” as used herein, refers to both quantitative as well as qualitative differences in the marker genes' temporal and/or cellular expression patterns within and among populations of immune cells, especially CLL cells.

In one specific embodiment of the invention, the marker gene is SEPT10, and an increase in the level of SEPT 10 expression in CLL cells relative to a reference level indicates a poor prognosis or an aggressive course of CLL. SEPT10 expression is correlated with the presence of unmutated immunoglobulin heavy chain variable regions. The SEPT10 gene encodes a member of the septin family of cytoskeletal proteins with GTPase activity. This protein localizes to the cytoplasm and nucleus and displays GTP-binding and GTPase activity (Sui et al., Biochem. Biophys. Res. Commun. 304 (2), 393-398 (2003)). Alternate splicing results in two transcript variants encoding different isoforms. The GenBank Accession Nos. related to nucleotide and amino acid sequences for SEPT10 isoform 1 are NM_{—}144710 and NP_{—}653311, respectively; and for SEPT10 isoform 2 are NM_{—}178584 and NP_{—}848699, respectively, which are all incorporated by reference in their entirety. The uses of all polynucleotides encoding the isoforms, variants, and polypeptides corresponding to the isoforms and variants, in the methods of the invention are encompassed. The term “SEPT10” is used collectively herein to refer to the coding regions and corresponding polypeptides of all isoforms of SEPT10. Examples of the sequence of a SEPT10 polynucleotide and a SEPT10 polypeptide are provided in SEQ ID NO: 1 and 2, respectively.

In another specific embodiment, the marker gene is KIAA0977, and an increase in the level of KIAA0977 expression in CLL cells relative to a reference level indicates a good prognosis or an indolent course of CLL. KIAA0977 expression in CLL cells is correlated with the presence of mutated immunoglobulin heavy chain variable regions. The term “KIAA0977” is used collectively herein to refer to the coding regions and corresponding polypeptides. The GenBank accession numbers related to KIAA0977 polynucleotides are AB023194, AL049939, and BC071588 (which encodes a human COBL-like protein). The uses of all polynucleotides encoding the variants, and polypeptides corresponding to the variants, in the methods of the invention are encompassed. Examples of the sequence of a KIAA0977 polynucleotide and a KIAA0977 polypeptide are provided in SEQ ID NO: 3 and 4, respectively.

In yet another specific embodiment, the marker gene is a hypothetical coding region located at Hs.23133, and an increase in the level of Hs.23133 expression in CLL cells relative to a reference level indicates a poor prognosis or an aggressive course of CLL. Hs.23133 expression is correlated with the presence of unmutated immunoglobulin heavy chain variable regions. The term “Hs.23133” is used herein collectively to refer to this hypothetical coding region and the corresponding hypothetical protein. Hs.23133 is a UniGene designation corresponding to LOC342935, mapped to 19q13.43, and encodes a hypothetical protein MGC9913. The GenBank accession numbers for the hypothetical mRNA sequence and the corresponding hypothetical protein amino acid sequence is XM_{—}378178.2 (and XM_{—}378178) and XM_{—}378718 respectively. Examples of the sequence of a Hs.23133 polynucleotide and a Hs.23133 polypeptide are provided in SEQ ID NO: 5 and 6, respectively.

In yet another specific embodiment, the marker gene is ADAM29, and an increase in the level of ADAM29 expression in CLL cells relative to a reference level indicates a good prognosis or an indolent course of CLL. ADAM29 expression in CLL cells is correlated with the presence of mutated immunoglobulin heavy chain variable regions. ADAM29 corresponds to Unigene designation Hs.126838 and encodes a disintegrin and metalloprotease domain 29. ADAM29 is a member of a protein family that include membrane-anchored proteins structurally related to snake venom disintegrins, and have been implicated in fertilization, muscle development and neurogenesis. Metalloproteinase-disintegrins (ADAMs) are type 1 transmembrane proteins that contain a unique domain structure including a zinc-binding metalloproteinase domain. ADAM29 is highly expressed in testis, and may be involved in spermatogenesis. ADAM29 is located at 4q34.2-qter. Alternative splicing generates 3 transcript variants which are divergent in the 3′ region, and encode proteins of 820, 786 and 767 amino acids. ADAM29-1 and ADAM29-2 share identical 228 bps in the 5′ end of coding region but differs in the 3′ end where ADAM29-1 is 33 amino acids longer than ADAM29-2 (see GenBank Accession Nos. AF134708 and AF171929 which are incorporated herein by reference in their entirety). ADAM29-3 (GenBank Accession No.: AF171930) has a deletion of 162 bp in the 3′ region compared to ADAM29-1. The uses of all polynucleotides encoding the variants, and polypeptides corresponding to the variants, in the methods of the invention are encompassed. See Xu et al., Genomics 1999, 62:537-9, which is incorporated herein by reference in its entirety. Examples of the sequence of an ADAM29 polynucleotide and a ADAM29 polypeptide are provided in SEQ ID NO: 7 and 8, respectively.

The methods of the invention may be performed using test cells in a sample derived from any tissue comprising CLL cells, including but not limited to spleen, lymph nodes, bone marrow, lymph, a whole blood sample from the subject, or a whole blood sample that has been processed to isolate the peripheral blood mononuclear cells (“PBMC”). In one embodiment, the sample may be enriched for CLL cells. In another embodiment, the sample may comprise purified CLL cells. In other embodiments, the sample may comprise test cells that are enriched for cells that express at least one of the following antigen: CD5, CD23, and/or CD19. In yet another embodiment, the test cells are enriched for naïve B cells, centroblasts, centrocytes, memory B cells, or T cells. In yet another embodiment, the test cells are purified cells that express at least one of the following antigen: CD5, CD23, and/or CD19; purified B cells, purified naïve B cells, purified centroblasts, purified centrocytes, or purified memory B cells.

According to the invention, a variety of molecular biological and immunological methods can be used to determine the level of marker gene expression in test cells. In one embodiment, the level of marker gene transcript in test cells is measured. In another embodiment, the level of marker protein or polypeptide in test cells is determined. In yet another embodiment, the percentage of CLL cells in a sample which are expressing the marker gene is determined.

Many embodiments of the invention are described hereinbelow generically using the term “marker” to denote any one of the four markers, SEPT10, KIAA0977, Hs.23133 and ADAM29. In specific embodiments where it is appropriate to identify the individual markers, such as when the markers behave differently in certain aspects, the invention is described by substituting the term “marker” with one of the names of the four markers.

As used herein, the phrases “marker polypeptide” and “marker protein” refer to a protein, polypeptide, peptide, and variants thereof, derived from a protein encoded by one of the genes or cDNAs of SEPT10, KIAA0977, Hs.23133 and ADAM29. These compositions are described in Section 5.2. Marker polypeptides encompass also polypeptides encoded by mRNA splice variants.

Nucleic acid molecules comprising nucleic acid sequences encoding the marker polypeptides or proteins of the invention, or genomic nucleic acid sequences from the marker genes (e.g., intron sequences, 5′ and 3′ untranslated sequences), or their complements thereof (i.e., antisense polynucleotides), are collectively referred to as “marker genes”, “marker polynucleotides” or “marker nucleic acid sequences” of the invention. The present invention also provides isolated marker polynucleotides or variants thereof, which can be used, for example, as hybridization probes or primers (“marker probes” or “marker primers”) to detect or amplify nucleic acids encoding a polypeptide of the invention. These compositions are described in Section 5.1.

The present invention also provides “marker antibodies”, including polyclonal, monoclonal, or recombinant antibodies, and fragments and variants thereof, that immunospecifically binds the respective marker proteins encoded by the genes or cDNAs (including polypeptides encoded by mRNA splice variants) of SEPT10, KIAA0977, Hs.23133 and ADAM29. Compositions comprising labeled marker polynucleotides, and labeled marker antibodies to the marker proteins or polypeptides are also encompassed by the invention. Marker antibodies are described in Section 5.3.

The invention further provides diagnostic reagents that depending on the techniques used in the assay method, comprise one or more marker probes, one or more marker primers, or one or more marker antibodies. A diagnostic reagent may comprise marker probes, marker primers or marker antibodies from the same marker gene or from multiple marker genes.

The invention also provides diagnostic compositions that comprise diagnostic reagents and a test subject's sample. Such diagnostic compositions are made whenever a method of the invention is carried out. Depending on the assay techniques used, a diagnostic composition may comprise, for example, marker probes and/or marker primers and target marker polynucleotides, or marker antibodies and target marker polypeptide, or marker antibodies and test cells. In many embodiments, the sample in a diagnostic composition is suspected of comprising a marker target polynucleotide, a marker polypeptide, or marker positive test cells. In some instances, a sample in a diagnostic composition may not comprise a detectable level of a marker target polynucleotide, a marker polypeptide, or marker positive test cells. These diagnostic compositions also yield useful information for the prognosis, and are encompassed by the invention. Despite the lack of a detectable signal, other nucleic acids and polypeptides that are characteristics of cells from a subject with CLL or that are not present in normal cells, are present in these samples. Accordingly, a diagnostic composition of the invention comprises one or more diagnostic reagents of the invention and a sample from a subject in need of a prognosis for CLL.

In many methods of the invention, the test subject's level of marker expression is compared against a reference level to provide the prognosis. For each different marker, test cell population, assay method, and reagent system, a different reference level is established using materials derived among CLL patients with a known clinical course and/or known IgV mutation status or cell lines of a known genotype/phenotype. In one embodiment, the reference level is based on statistics obtained from a characterized patient population, such as a large diverse population. A reference level can also be established for different age groups, ethnicity, and/or gender. It is contemplated that the numerical cut off value for a reference level that defines poor or favorable prognosis may be shifted upward or downward with a possible loss of accuracy. However, it is well within the skill of one of ordinary skill in the art to determine the appropriate reference level, by either using the experimental methods disclosed herein, or by comparing the relative specificity and sensitivity of the reagents used in the methods and taking into consideration the variable parameters. For example, for each marker, different reference levels may be used depending on the purity of the CLL cells, the specific anti-CD5 and anti-CD19 antibodies used, and the anti-CD5 and anti-CD19 fluorochrome conjugates used.

For clarity of disclosure, and not by way of limitation, the detailed description of the invention is divided into the subsections that follow. Nucleic acid-based assay methods are described in Section 5.4.1 and protein-based assay methods, including cell-based methods are described in Section 5.4.2. As used herein, “a” or “an” means at least one, unless clearly indicated otherwise.

### 5.1 Marker Polynucleotides

The present invention provides four sets of marker polynucleotides and their uses in various assay methods. Also provided are diagnostic reagents and compositions comprising one or more marker polynucleotides. The four sets of marker polynucleotides are derived from the four marker genes, SEPT10, KIAA0977, Hs.23133 and ADAM29. The term polynucleotide as used herein is intended to include DNA molecules (e.g., cDNA, genomic DNA), RNA molecules (e.g., hnRNA, pre-mRNA, mRNA), and DNA or RNA analogs generated using nucleotide and/or nucleoside analogs. The polynucleotide can be single-stranded or double-stranded. An isolated polynucleotide is one which is distinguished from other polynucleotides that are present in the natural source of the polynucleotide. An isolated polynucleotide, such as a cDNA molecule, can be substantially free of other cellular material, or culture medium when produced by recombinant techniques, or substantially free of chemical precursors or other chemicals when chemically synthesized.

An isolated marker polynucleotide can comprise flanking sequences (i.e., sequences located at the 5′ or 3′ ends of the nucleic acid), which naturally flank the nucleic acid sequence in the genomic DNA of the organism from which the nucleic acid is derived. However, an isolated polynucleotide does not include an isolated chromosome, and does not include the poly(A) tail of an mRNA, if present. For example, in various embodiments, the isolated marker polynucleotide can comprise less than about 5 kb, 4 kb, 3 kb, 2 kb, 1 kb, 0.5 kb or 0.1 kb of nucleotide sequences which naturally flank the coding sequence in genomic DNA of the cell from which the nucleic acid is derived. In other embodiments, the isolated marker polynucleotide is about 10-20, 21-50, 51-100, 101-200, 201-400, 401-750, 751-1000, 1001-1500 bases in length.

In various embodiments, the marker polynucleotides of the invention are used as molecular probes in hybridization reactions or as molecular primers in nucleic acid extension reactions. In these instances, the marker polynucleotides may be referred to as marker probes and marker primers, respectively, and the marker polynucleotides present in a sample which are to be detected and/or quantified are referred to as target marker polynucleotides. Two marker primers are commonly used in DNA amplification reactions and they are referred to as marker forward primer and marker reverse primer depending on their 5′ to 3′ orientation relative to the direction of transcription. A marker probe or a marker primer is typically an oligonucleotide which binds through complementary base pairing to a subsequence of a target marker polynucleotide. The marker probe may be, for example, a DNA fragment prepared by amplification methods such as by PCR or it may be chemically synthesized. A double stranded fragment may then be obtained, if desired, by annealing the chemically synthesized single strands together under appropriate conditions or by synthesizing the complementary strand using DNA polymerase with an appropriate primer. Where a specific nucleic acid sequence is given, it is understood that the complementary strand is also identified and included as the complementary strand will work equally well in situations where the target is a double stranded nucleic acid. A nucleic acid probe is complementary to a target nucleic acid when it will anneal only to a single desired position on that target nucleic acid under proper annealing conditions which depend, for example, upon a probe's length, base composition, and the number of mismatches and their position on the probe, and must often be determined empirically. Such conditions can be determined by those of skill in the art.

In one embodiment, the invention provides diagnostic reagents that comprise one or more marker probes, or one or more marker primers. A diagnostic reagent may comprise marker probes and/or marker primers from the same marker gene or from multiple marker genes. In another embodiment, the invention also provides diagnostic compositions that comprise one or more marker probes and target marker polynucleotides, or one or more marker primers and target polynucleotides, or marker primers, marker probes and marker target polynucleotides. In some embodiments, the diagnostic compositions comprise marker probes and/or marker primers and a sample suspected to comprise marker target polynucleotides. Such diagnostic compositions comprise marker probes and/or marker primers and the nucleic acid molecules (including RNA, mRNA, cRNA, cDNA, and/or genomic DNA) of a subject in need of a prognosis of CLL.

Depending on the reaction conditions, the marker probes or primers and target polynucleotides may form molecular complexes by nucleic acid hybridization in the diagnostic compositions. If nucleic acid amplification is involved, a diagnostic composition may also comprise extended double-stranded and/or single-stranded nucleic acid molecules comprising one or more marker primers. The extensions comprise nucleic acids corresponding to segments of a target polynucleotide. In one embodiment, the marker primers and marker probes are purified; and the diagnostic reagents and compositions comprise purified marker primers and/or purified marker probes.

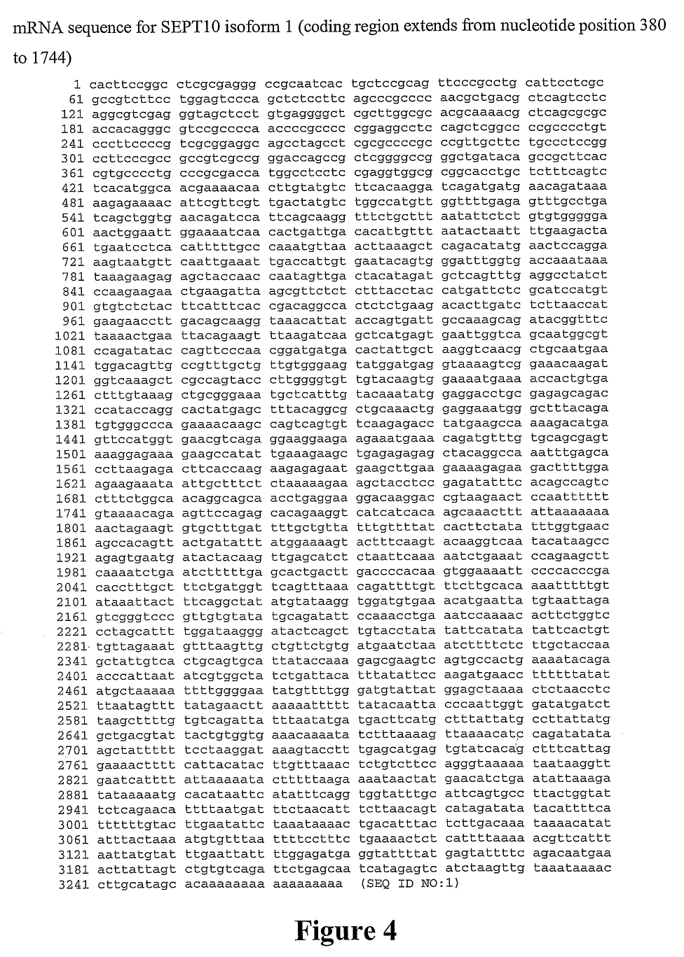

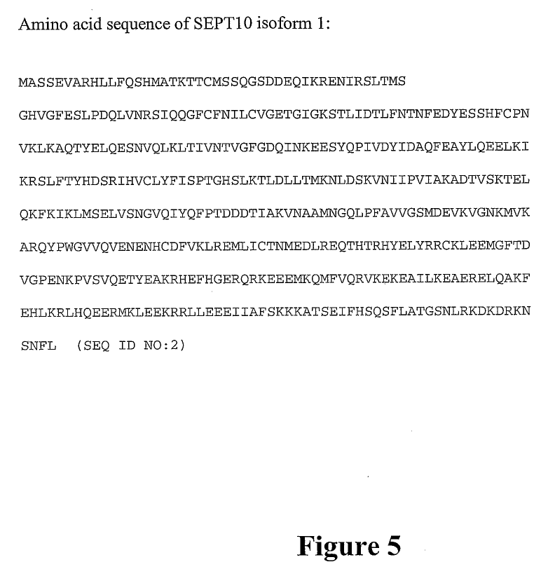

Accordingly, in one embodiment, the invention provides SEPT10 polynucleotides which encompass (a) a nucleic acid molecule comprising the DNA sequence shown in SEQ ID NO: 1 (FIG. 4) or Genebank Accession No. NM_{—}144710; (b) any nucleic acid molecule comprising a DNA sequence that encodes the amino acid sequence shown in SEQ ID NO:2 (FIG. 5) or GenBank Accession No. NP_{—}653311; (c) a nucleic acid molecule comprising the complement of the DNA sequences that encode the amino acid sequence shown in SEQ ID NO:2 or in GenBank Accession No. NP_{—}653311; (d) a nucleic acid molecule that hybridizes to another nucleic acid consisting of the DNA sequence that encodes the amino acid sequence shown in SEQ ID NO:2 or in GenBank Accession No. NP_{—}653311, under highly stringent conditions, e.g., hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (Ausubel F. M. et al., eds., 1989, Current Protocols in Molecular Biology, Vol. I, Green Publishing Associates, Inc., and John Wiley & sons, Inc., New York, at page 2.10.3); and (e) a nucleic acid molecule that hybridizes to another nucleic acid consisting of the complement of a DNA sequence that encodes the amino acid sequence shown in SEQ ID NO:2 or in GenBank Accession No. NP_{—}653311, under highly stringent conditions. The term SEPT10 polynucleotide as used herein includes nucleic acid encoding all isoforms of SEPT10, e.g., NP_{—}848699 which is encoded by NM_{—}178584. The invention also provides SEPT10 probes, SEPT10 primers, and SEPT10 target polynucleotides, SEPT10 diagnostic reagents comprising SEPT10 probes and/or SEPT10 primers, and SEPT10 diagnostic compositions comprising SEPT10 probes and/or SEPT10 primers, and target SEPT10 polynucleotides or samples suspected to comprise target SEPT10 polynucleotides.

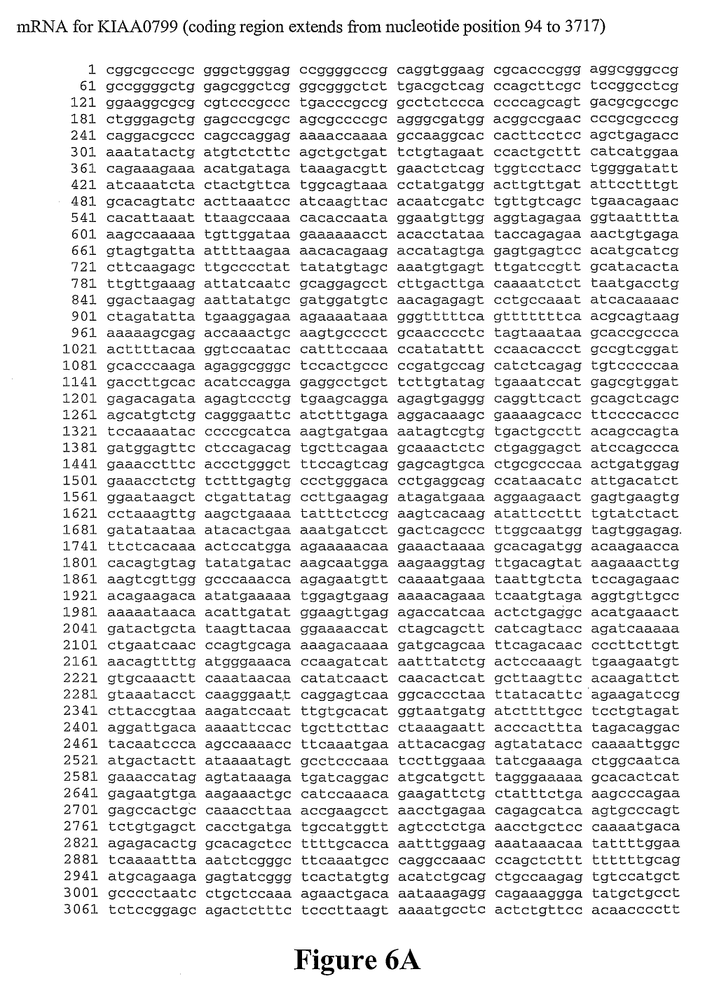

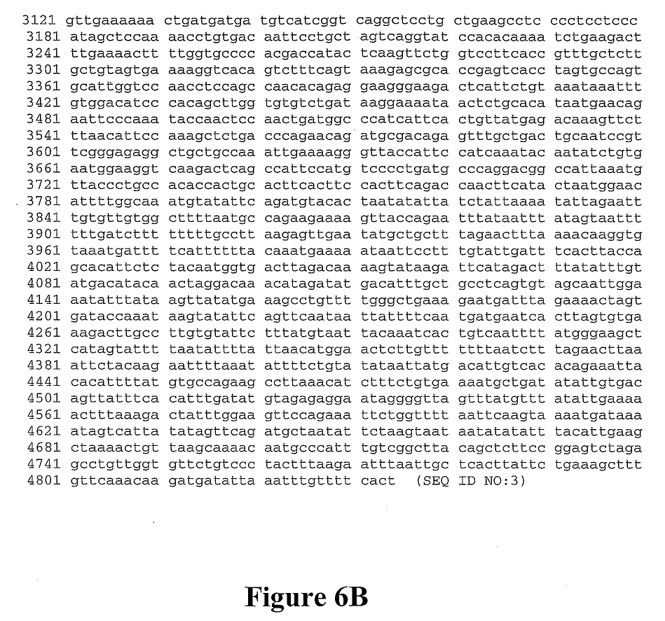

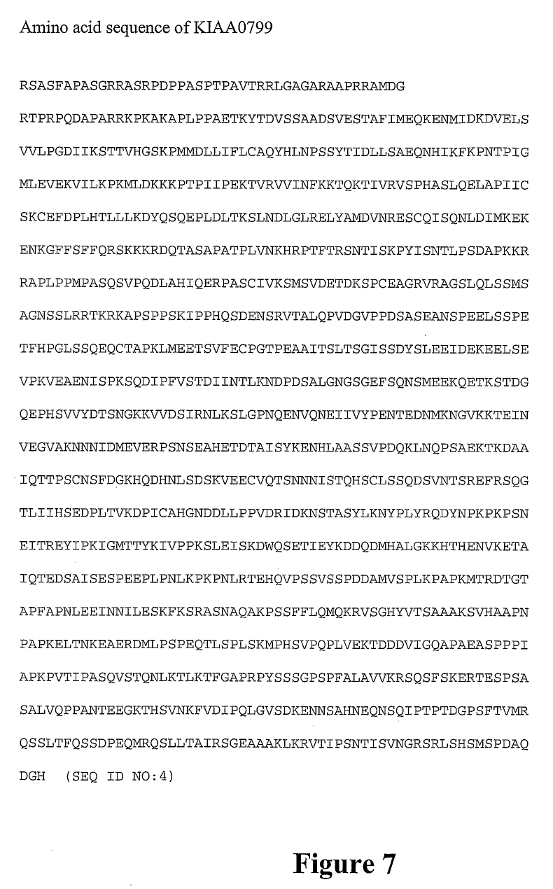

In another embodiment, the invention provides KIAA0977 polynucleotides which encompass (a) a nucleic acid molecule comprising the DNA sequence shown in SEQ ID NO:3 (FIGS. 6A-6B) or Genebank Accession No. AB023194; (b) any nucleic acid molecule comprising a DNA sequence that encodes the amino acid sequence shown in SEQ ID NO:4 (FIG. 7) or GenBank Accession No. AB023194; (c) a nucleic acid molecule comprising the complement of the DNA sequences that encode the amino acid sequence shown in SEQ ID NO:4 or in GenBank Accession No. AB023194; (d) a nucleic acid molecule that hybridizes to another nucleic acid consisting of the DNA sequence that encodes the amino acid sequence shown in SEQ ID NO:4 or in GenBank Accession No. AB023194, under highly stringent conditions; and (e) a nucleic acid molecule that hybridizes to another nucleic acid consisting of the complement of a DNA sequence that encodes the amino acid sequence shown in SEQ ID NO:4 or in GenBank Accession No. AB023194, under highly stringent conditions. The invention also provides KIAA0977 probes, KIAA0977 primers, and KIAA0977 target polynucleotides, KIAA0977 diagnostic reagents comprising KIAA0977 probes and/or KIAA0977 primers, and KIAA0977 diagnostic compositions comprising KIAA0977 probes and/or KIAA0977 primers, and KIAA0977 target polynucleotides or samples suspected to comprise KIAA0977 target polynucleotides.

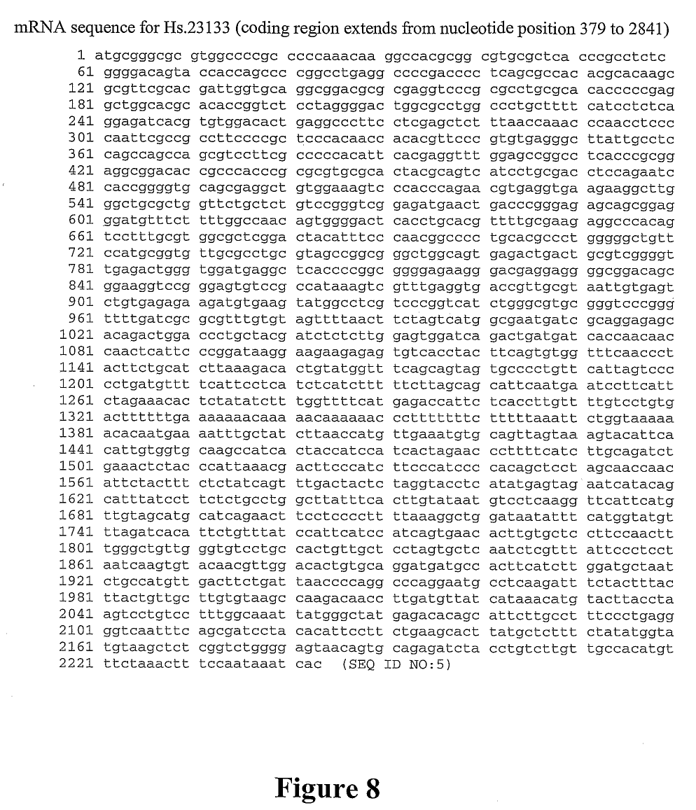

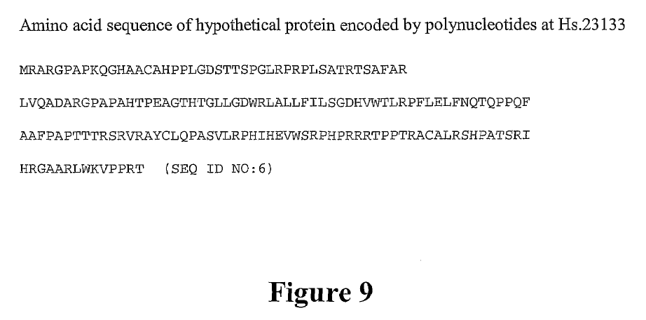

In yet another embodiment, the invention provides Hs.23133 polynucleotides which encompass (a) a nucleic acid molecule comprising the DNA sequence shown in SEQ ID NO:5 (FIG. 8) or Genebank Accession No. XM_{—}378178.2; (b) any nucleic acid molecule comprising a DNA sequence that encodes the amino acid sequence shown in SEQ ID NO:6 (FIG. 9) or GenBank Accession No. XP_{—}378718; (c) a nucleic acid molecule comprising the complement of the DNA sequences that encode the amino acid sequence shown in SEQ ID NO:6 or in GenBank Accession No. XP_{—}378718; (d) a nucleic acid molecule that hybridizes to another nucleic acid consisting of the DNA sequence that encodes the amino acid sequence shown in SEQ ID NO: 6 or in GenBank Accession No. XP_{—}378718, under highly stringent conditions; and (e) a nucleic acid molecule that hybridizes to another nucleic acid consisting of the complement of a DNA sequence that encodes the amino acid sequence shown in SEQ ID NO:6 or in GenBank Accession No. XP_{—}378718, under highly stringent conditions. The invention also provides Hs.23133 probes, Hs.23133 primers, and Hs.23133 target polynucleotides, Hs.23133 diagnostic reagents comprising Hs.23133 probes and/or Hs.23133 primers, and Hs.23133 diagnostic compositions comprising Hs.23133 probes and/or Hs.23133 primers, and Hs.23133 target polynucleotides or samples suspected to comprise Hs.23133 target polynucleotides.

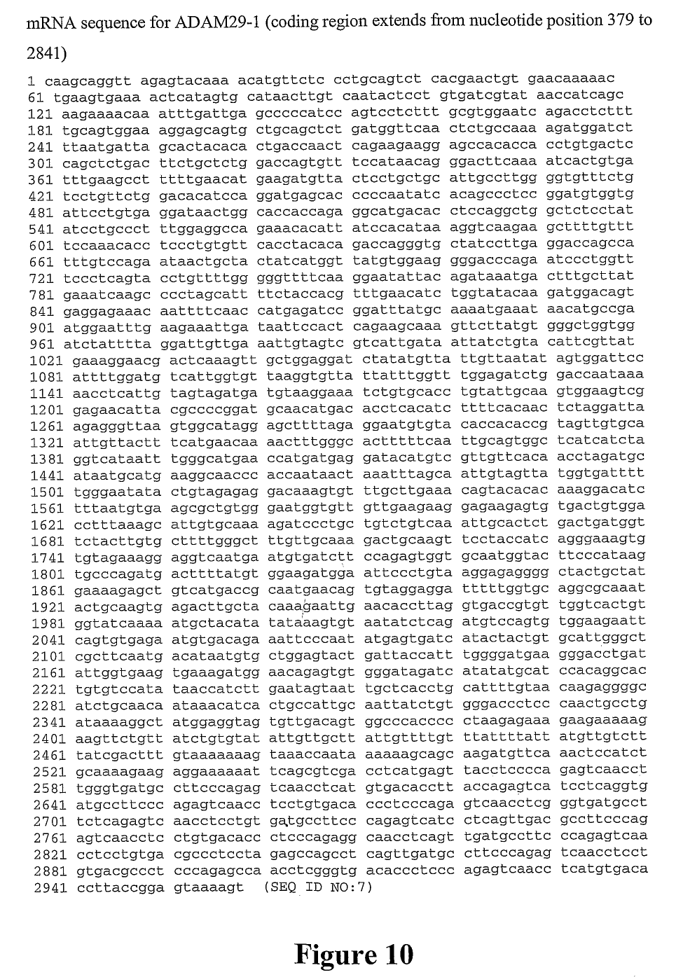

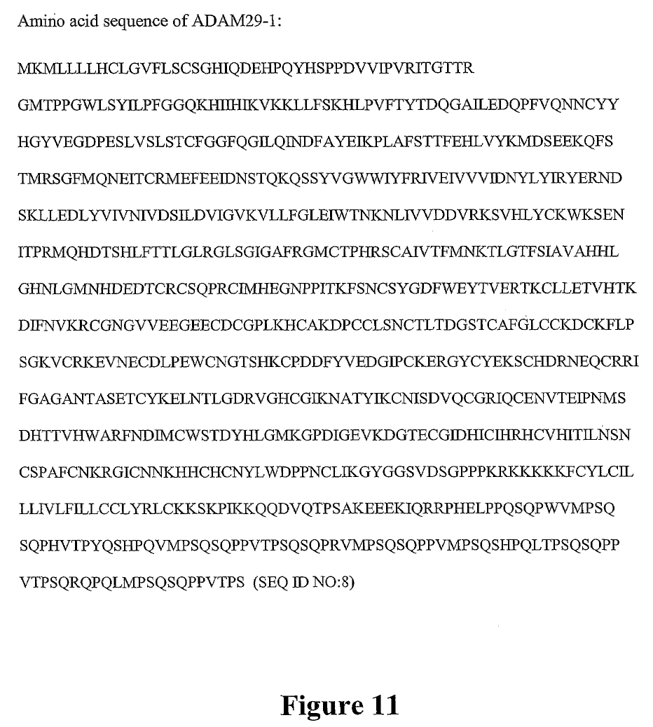

In yet another embodiment, the invention provides ADAM29 polynucleotides which encompass (a) a nucleic acid molecule comprising the DNA sequence shown in SEQ ID NO:7 (FIG. 10) or Genebank Accession No. AF134708; (b) any nucleic acid molecule comprising a DNA sequence that encodes the amino acid sequence shown in SEQ ID NO:8 (FIG. 11) or GenBank Accession No. AF134708; (c) a nucleic acid molecule comprising the complement of the DNA sequences that encode the amino acid sequence shown in SEQ ID NO:8 or in GenBank Accession No. AF134708; (d) a nucleic acid molecule that hybridizes to another nucleic acid consisting of the DNA sequence that encodes the amino acid sequence shown in SEQ ID NO:8 or in GenBank Accession No. AF134708, under highly stringent conditions; and (e) a nucleic acid molecule that hybridizes to another nucleic acid consisting of the complement of a DNA sequence that encodes the amino acid sequence shown in SEQ ID NO:8 or in GenBank Accession No. AF134708, under highly stringent conditions. The term ADAM29 polynucleotide as used herein includes nucleic acids encoding all variants of ADAM29, e.g., AF171929 and AF171930. The invention also provides ADAM29 probes, ADAM29 primers, and ADAM29 target polynucleotides, ADAM29 diagnostic reagents comprising ADAM29 probes and/or ADAM29 primers, and ADAM29 diagnostic compositions comprising ADAM29 probes and/or ADAM29 primers, and ADAM29 target polynucleotides or samples suspected to comprise ADAM29 target polynucleotides.

The term “hybridizes under highly stringent conditions” as exemplified above is intended to describe generally conditions for hybridization and washing under which nucleotide sequences that are at least about 60%, about 65%, about 70%, or about 75% identical to each other typically remain hybridized to each other. Many such stringent conditions are known to those skilled in the art and examples can be found in Current Protocols in Molecular Biology, John Wiley & Sons, N.Y. (1989) pp. 6.3.1-6.3.6. Another non-limiting example of stringent hybridization conditions are hybridization in 6× sodium chloride/sodium citrate (“SSC”) at about 45° C. followed by one or more washes in 0.2×SSC, 0.1% SDS at 50-65° C.

As used herein, the term “variant” or “variants” refers to, where appropriate, variations of the nucleic acid or amino acid sequence of marker molecules such as, but not limited to, homologs, analogs, derivatives, fragments, hybrids, mimetics, congeners, and nucleotide and amino acid substitutions, additions, deletions, or other chemical modifications.

In one embodiment, a variant marker probe hybridizes to a naturally-occurring target polynucleotide under stringent conditions. In another embodiment, a variant marker probe hybridizes to a naturally-occurring target polynucleotide under moderately stringent conditions. The present invention also provides isolated polynucleotides encoding a variant marker polypeptide. An isolated polynucleotide that encodes a variant polypeptide can be created by introducing one or more nucleotide substitutions, additions or deletions into the nucleotide sequence of the marker gene using any method known in the art.

Further, the present invention encompasses marker polynucleotide that are specific portions of a full length marker polynucleotide or marker polypeptide that can be discerned as a domain or motif such as, for example, a portion of a marker polynucleotide or polypeptide having a predicted biological activity. Such domains and motifs include, but are not limited to, exons, introns, splice acceptor sites, splice donor sites, 5′ regulatory regions of the mRNA, 3′ regulatory regions of the mRNA, mRNA capping regions, promoter regions, transcriptional regulatory sites, enhancer sequences, glycosylation sites, ligand-binding sites, and variants thereof. Accordingly, a polynucleotide encoding such motifs or domains is encompassed by the marker polynucleotides of the invention, and any polypeptide encoded by such marker polynucleotides is encompassed by the marker polypeptides of the invention. Accordingly, a marker polynucleotide can comprise cDNA, genomic DNA, introns, exons, promoter regions, 5′ regulatory regions of the gene, 3′ regulatory regions of the gene, RNA, hnRNA, mRNA, regulatory regions within RNAs, and variants thereof.

For many methods of the invention, it is not necessary to use a marker polynucleotide that comprises the entire coding region or the entire mRNA. Accordingly, in other embodiments of the invention, the diagnostic reagents comprise a marker polynucleotide which does not consist of the entire nucleotide sequence disclosed in any one of the following GenBank accession numbers: NM_{—}144710, NM_{—}17854, AB023194, AL049939, BC071588, XM_{—}378178.2, AF134708, AF171929, and AF171930.

Using all or a portion of the nucleic acid sequences of SEQ ID NO: 1, 3, 5, or 7, as a hybridization probe, polynucleotides of the invention can be isolated using standard hybridization and cloning techniques (See, e.g., Sambrook et al., Molecular Cloning: A Laboratory Manual, 2nd ed., Cold Spring Harbor Laboratory, Cold Spring Harbor Laboratory Press, Cold Spring Harbor, N.Y., 1989).

In various embodiments of the invention, a polynucleotide sequence encoding a marker polypeptide is inserted into an expression vector for propagation and expression in recombinant cells. Many methods known in the art can be used to produce marker polypeptides and modified marker polypeptides, including but not limited to fusion proteins, fragments and derivatives thereof. An expression construct, as used herein, refers to a nucleotide sequence encoding a marker polypeptide operably associated with one or more regulatory regions which enables expression of the marker polypeptide in an appropriate host cell. “Operably-associated” refers to an association in which the regulatory regions and the marker polynucleotide to be expressed are joined and positioned in such a way as to permit transcription, and ultimately, translation.

### 5.2 Marker Polypeptides

The present invention also provides marker polypeptides, including peptides, and variants thereof, derived from a protein encoded by one of the genes or cDNAs of SEPT10, KIAA0977, Hs.23133 and ADAM29 as described in the previous section.

In one embodiment, a marker polypeptide can be used to generate diagnostic reagents, such as binding partners, and marker antibodies. For such uses, the marker polypeptide can be purified or isolated. As used herein, an isolated or purified marker protein or a portion thereof is substantially free of cellular material or other contaminating proteins from the cell or tissue source from which the marker protein is derived, or substantially free of chemical precursors or other chemicals when chemically synthesized. The language “substantially free” indicates protein preparations in which the protein is separated from cellular components of the cells from which it is isolated or recombinantly produced. Thus, protein that is substantially free of cellular material includes protein preparations having less than 20%, 10%, or 5% (by dry weight) of a contaminating protein. Similarly, when an isolated marker polypeptide of the invention is recombinantly produced and isolated, it is substantially free of culture medium. When the marker polypeptide is produced by chemical synthesis, it is substantially free of chemical precursors or other chemicals. Such marker polypeptides can also be used as positive controls in the assays of the invention.

In another embodiment, the invention provides marker polypeptides that are the target of polypeptide-based diagnostic assays of the invention. Such target marker polypeptides are present in cells, located in various subcellular compartments, or embedded in the membrane. Depending on the assay method, the marker polypeptide may be purified, enriched in a cell extract or fraction, or unpurified in a cell, in a permeabilized cell, in a cell membrane, or on a cell surface. Marker polypeptides of the invention can comprise, for example, a extracellular domain, transmembrane domain, intracellular domain, signal peptide, phosphorylation sites, glycosylation signals, subcellular localization signals, or protein degradation signals. Diagnostically relevant portions of a marker protein of the invention comprise amino acid sequences identical to or derived from the amino acid sequence of a marker protein, including variants sequences comprising fusions or truncations (e.g., amino acid sequences comprising fewer amino acids than those shown in any of SEQ ID NO: 2, 4, 6, and 8, but which maintain a high degree of homology to the remaining amino acid sequence). Such fusions or truncations may be the result of chromosomal aberration. A diagnostically relevant portion of a marker protein of the invention can be a polypeptide which is, for example, at least 25, 50, 100, 200, 300, 400 or 500 amino acids in length.

In other embodiments, marker polypeptides consist of the amino acid sequence of SEQ ID NO: 2, 4, 6, or 8. Other useful polypeptides are substantially identical (e.g., at least about 65%, about 75%, about 85%, about 90%, about 95%, or about 99%) to any of SEQ ID NO: 2, 4, 6, and 8. In other embodiments, the invention provides fragments of the amino acid sequence wherein the percent identity is determined over amino acid sequences of identical size to the fragment. In other embodiments, the invention provides a polypeptide comprising an amino acid sequence that has at least 90% identity to the fragments of domains identified in the marker polypeptides, wherein the percent identity is determined over an amino acid sequence of identical size to said fragment.

The invention also provides chimeric or fusion proteins. As used herein, a “chimeric protein” or “fusion protein” comprises all or part of a marker polypeptide of the invention fused in-frame to a second polypeptide. In one embodiment, the second polypeptide is a heterologous polypeptide. In another embodiment, the second polypeptide is different from, but derived from the same, polypeptide to which it is attached. The second polypeptide can be fused to the N-terminus or C-terminus of the polypeptide of the invention. Such fusion proteins can be a by-product of a chromosomal aberration present in leukemic cells.

For example, one useful fusion protein is a GST fusion protein in which the polypeptide of the invention is fused to the C-terminus of GST sequences. Such fusion proteins can facilitate the purification of a recombinant polypeptide of the invention. Chimeric and fusion proteins of the invention can be produced by standard recombinant DNA techniques. In one embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, e.g., Ausubel et al., supra). Moreover, many expression vectors are commercially available that already encode a fusion moiety (e.g., a GST polypeptide). A nucleic acid encoding a polypeptide of the invention can be cloned into such an expression vector such that the fusion moiety is linked in-frame to the polypeptide of the invention.

The present invention also pertains to variants of the polypeptides of the invention. Such variants have an altered amino acid sequence which can either be made synthetically, arise as a result of alternative splicing, or detected in CLL patients as a result of a chromosomal aberration. The marker polypeptides of the invention can exhibit post-translational modifications, including, but not limited to glycosylations, (e.g., N-linked or O-linked glycosylations), myristylations, palmitylations, acetylations and phosphorylations (e.g., serine/threonine or tyrosine).

Accordingly, the invention encompasses SEPT10 polypeptides, KIAA0977 polypeptides, Hs.23133 polypeptides, and ADAM29 polypeptides, as well as, the respective fusion proteins, fragments, variants, derivatives thereof. Also encompassed in the invention are binding partners of the marker polypeptides. As used herein, a binding partner binds specifically to a marker polypeptide and encompasses antibodies to the marker polypeptide, naturally occurring cofactors or substrates of the marker polypeptide. In one embodiment, the binding partner is an antibody as described in the next section. In specific embodiments, the invention provides diagnostic compositions that comprise one or more markers binding partners and target marker polypeptides. In some embodiments, the diagnostic compositions comprise marker binding partners and a sample suspected to comprise marker target polypeptides. A diagnostic composition may comprise diagnostic reagent and a sample of a subject in need of prognosis for CLL and that comprises a negligible amount of a target marker polypeptide.

### 5.3 Marker Antibodies

In various embodiments of the invention, marker antibodies as well as fragments, derivatives or analogs thereof can be used in the methods of the invention for determining the prognosis of B cell chronic lymphocytic leukemia. The term “antibody” as used herein is meant to include polyclonal antibodies, monoclonal antibodies, chimeric antibodies and single chain antibodies. In one embodiment, the antibodies are monoclonal antibodies, which may be of any immunoglobulin class including IgG, IgM, IgE, IgD, IgA, IgY and any subclass or isotype thereof (e.g., IgG₁, IgG₂, IgG₃, IgG₄, IgA₁, and IgA₂). The term “antibody” is also meant to include both intact immunoglobulin molecules as well as fragments thereof which bind immunospecifically to a marker polypeptide or protein, such as, for example, F(ab′)2, Fab′, Fab, Fv, single-chain Fvs (scFv, including bi-specific scFvs), and disulfide-linked Fvs (sdFv). Many such fragments can be produced by proteolytic cleavage, using enzymes such as papain (to produce Fab fragments) or pepsin (to produce F(ab′)₂ fragments) or by reducing the disulfide bridges. Some are produced by recombinant DNA techniques.

An isolated marker polypeptide of the invention, SEPT10, KIAA0977, Hs.23133 or ADAM29, or a fragment thereof, can be used as an immunogen to generate antibodies using standard techniques for polyclonal and monoclonal antibody preparation. The full-length marker polypeptide or protein can be used or, alternatively, the invention provides antigenic peptide fragments for use as immunogens. In one embodiment, the antigenic peptide of a marker protein of the invention comprises at least 8, 10, 15, 20, or 30 consecutive amino acid residues of the amino acid sequence of SEQ ID NO:2, 4, 6, or 8, and encompasses an epitope of the marker protein such that an antibody raised against the peptide forms a specific immune complex with the protein. In other embodiments, epitopes are regions that are located on the surface of the protein, e.g., hydrophilic regions. Publically available software can be used for selecting segments of a protein for maximum antigenicity.

Accordingly, the invention provides in various embodiments, anti-SEPT10 antibodies, anti-KIAA0977 antibodies, anti-Hs.23133 antibodies, and anti-ADAM29 antibodies. Also provided are diagnostic reagents comprising one or more of anti-SEPT10 antibodies, anti-KIAA0977 antibodies, anti-Hs.23133 antibodies, and anti-ADAM29 antibodies; and diagnostic compositions comprising one or more of anti-SEPT10 antibodies, anti-KIAA0977 antibodies, anti-Hs.23133 antibodies, and anti-ADAM29 antibodies, and a sample comprising one or more of the four marker proteins, or a sample suspected of comprising one or more of the four marker proteins. In specific embodiments, the invention provides substantially purified antibodies or fragments thereof, which antibodies or fragments specifically bind to a marker polypeptide of the invention comprising an amino acid sequence selected from the group consisting of: the amino acid sequence of SEQ ID NO:2, 4, 6, or 8; a fragment of at least 8 contiguous amino acid residues of the amino acid sequence of SEQ ID NO:2, 4, 6, or 8; an amino acid sequence which is at least 95% identical to the amino acid sequence of SEQ ID NO:2, 4, 6, or 8, wherein the percent identity is determined using the ALIGN program of the GCG software package with a PAM120 weight residue table, a gap length penalty of 12, and a gap penalty of 4.

An immunogen is used to prepare marker antibodies by immunizing a suitable animal, typically a mammal or a bird, such as goat, mouse, sheep, horse, chicken, rabbit, guinea pig, or rat. An appropriate immunogenic preparation can comprise, for example, purified marker polypeptide, recombinantly expressed or chemically synthesized marker polypeptide. The preparation can further include an adjuvant, such as Freud's complete or incomplete adjuvant, or similar immunostimulatory agent including but not limited to mineral gels such as aluminum hydroxide, surface active substances such as lysolecithin, pluronic polyols, polyanions, peptides, oil emulsions, keyhole limpet hemocyanin, dinitrophenol, and human adjuvants such as BCG (bacille Calmette-Guerin) and Corynebacterium parvum. The antibody titer in the immunized subject can be monitored over time by standard techniques, such as with an enzyme linked immunosorbent assay (ELISA) using immobilized polypeptide.

If desired, the antibody molecules can be isolated from the mammal (e.g., from the blood) and further purified by well-known techniques, such as protein A chromatography to obtain the IgG fraction. Alternatively, antibodies specific for a marker polypeptide of the invention can be selected for (e.g., partially purified) or purified by, e.g., affinity chromatography. For example, a recombinantly expressed and purified (or partially purified) marker protein of the invention is produced by standard recombinant DNA techniques, and covalently or non-covalently coupled to a solid support such as, for example, a chromatography column. The column can then be used to affinity purify antibodies specific for the proteins of the invention from a sample comprising antibodies directed against a large number of different epitopes, thereby generating a substantially purified antibody composition, i.e., one that is substantially free of contaminating antibodies. By a substantially purified antibody composition is meant, in this context, that the antibody sample comprises at most only about 30%, about 20%, about 10%, or about 5% (by dry weight) of contaminating antibodies directed against epitopes other than those on the desired protein or polypeptide of the invention. A purified antibody composition means that at least about 99% of the antibodies in the composition are directed against the desired protein or polypeptide of the invention.

At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein (1975, Nature 256:495-497), the human B cell hybridoma technique (Kozbor et al., 1983, Immunol Today 4:72), the EBV-hybridoma technique (Cole et al., 1985, in Monoclonal Antibodies and Cancer Therapy, Alan R. Liss, Inc., pp. 77-96) or trioma techniques. The technology for producing hybridomas is well known (See, e.g., Current Protocols in Immunology, Coligan et al. (eds.) John Wiley & Sons, Inc., New York, N.Y. (1994)). Hybridoma cells producing a monoclonal antibody of the invention are detected by screening the hybridoma culture supernatants for antibodies that bind the polypeptide of interest, e.g., using a standard ELISA assay. The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Alternative to preparing monoclonal antibody-secreting hybridomas, a monoclonal antibody directed against a polypeptide of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening an antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, BioTechnology 9:1370-1372; Hay et al., 1992, Hum Antibod Hybridomas 3:81-85; Huse et al., 1989, Science 246:1275-1281; Griffiths et al., 1993, EMBO J. 12:725-734.

Additionally, recombinant antibodies, such as chimeric and humanized monoclonal antibodies, comprising both human and non-human portions, which can be made using standard recombinant DNA techniques, are within the scope of the invention. A chimeric antibody is a molecule in which different portions are derived from different animal species, such as those having a variable region derived from a murine monoclonal antibody and a human immunoglobulin constant region (see, e.g., Cabilly et al., U.S. Pat. No. 4,816,567; and Boss et al., U.S. Pat. No. 4,816,397, which are incorporated herein by reference in their entirety). Humanized antibodies are antibody molecules from non-human species having one or more complementarity determining regions (CDRs) from the non-human species and a framework region from a human immunoglobulin molecule (see, e.g., Queen, U.S. Pat. No. 5,585,089, which is incorporated herein by reference in its entirety). Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al., 1988, Science 240:1041-1043; Liu et al., 1987, Proc Natl Acad Sci. 84:3439-3443; Liu et al., 1987, J. Immunol. 139:3521-3526; Sun et al., 1987, Proc Natl Acad Sci. 84:214-218; Nishimura et al., 1987, Cancer Res. 47:999-1005; Wood et al., 1985, Nature 314:446-449; and Shaw et al., 1988, J. Natl. Cancer Inst. 80:1553-1559; Morrison, 1985, Science 229:1202-1207; Oi et al., 1986, BioTechniques 4:214; U.S. Pat. No. 5,225,539; Jones et al., 1986, Nature 321:552-525; Verhoeyan et al., 1988, Science 239:1534; and Beidler et al., 1988, J. Immunol. 141:4053-4060. Single chain antibodies are formed by linking the heavy and light chain fragments of the Fv region via an amino acid bridge, resulting in a single chain polypeptide (see, e.g., Pantoliano et al., 1991, Biochemistry 30(42):10117-25). Accordingly, the present invention provides the nucleotide and deduced amino acid sequences of a purified monoclonal marker antibodies. Such sequences allow for the production of recombinant forms of the marker antibodies (e.g., human, humanized, chimerized and/or tolerized forms), as well as genetically engineered fragments thereof (e.g., single-chain Fvs (scFv) (including bi-specific scFvs), single chain antibodies, Fab fragments, F(ab′) fragments, F(ab′)₂ fragments, disulfide-linked Fvs (sdFv) and fragments thereof, and epitope-binding fragments of any of the above.

The marker antibodies of the present invention may also be described or specified in terms of their binding affinity to one of the marker polypeptide or a portion thereof: SEPT10, KIAA0977, Hs23133 and ADAM29. In other embodiments, binding affinities include those with a dissociation constant or Kd less than 5×10⁻² M, 10⁻² M, 5×10⁻³ M, 10⁻³ M, 5×10⁻⁴ M, 10⁻⁴ M, 5×10⁻⁵ M, 10⁻⁵M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

In various embodiments, the antibodies used in the methods of the invention include derivatives that are modified, i.e, by the covalent attachment of any type of molecule to the antibody. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

The antibodies of the invention are reactive to the marker polypeptides of the invention. In specific embodiments, an antibody directed against a marker polypeptide of the invention can be used to detect the presence of a marker polypeptide in a sample in order to evaluate the abundance and pattern of expression of the polypeptide. The antibodies can also be used diagnostically to monitor marker levels in cells and/or tissues as part of a clinical testing procedure, for example, to provide a prognosis or to determine the efficacy of a given treatment regimen. It is contemplated that a plurality of marker antibodies directed to different marker polypeptides can be used as in combination as a panel for various methods of the invention.

In various embodiments, the invention provides diagnostic reagents that comprise one or more marker antibodies. A diagnostic reagent may comprise marker antibodies from the same marker gene or from multiple marker genes. In many embodiments, a marker antibody in a diagnostic reagent is designed to be used in conjunction with other reagents in a reagent system, which can be provided in the form of a kit. For example, a marker antibody raised in a first species of animal can be used with secondary antibodies which specifically bind to antibodies of the first species. A marker antibody can be indirectly labeled by such secondary antibodies that are labeled.

In another embodiment, the invention also provides diagnostic compositions that comprise one or more marker antibodies and target marker polypeptides, or one or more marker antibodies and test cells comprising target marker polypeptides. In some embodiments, the diagnostic compositions comprise marker antibodies and a sample suspected to comprise marker target polypeptides. In other embodiments, such diagnostic compositions comprise marker antibodies and purified marker polypeptides, or compositions comprising marker polypeptides. In other embodiments, such diagnostic compositions comprise test cells comprising CLL cells, cell suspensions, cell extracts, or cell fractions from a subject in need of a prognosis of CLL. In other embodiments, the test cells and related cell compositions in such diagnostic compositions are enriched for CLL cells or comprise purified CLL cells. Test cell populations enriched for CLL cells may be isolated or sorted by expression of at least one of the following antigens: CD5, CD23 and CD19.

Various chemical or biochemical derivatives of the antibodies or antibody fragments of the present invention can be produced using known methods. One type of derivative which is diagnostically useful is an immunoconjugate comprising an antibody molecule, or an antigen-binding fragment thereof, to which is conjugated a detectable label. However, in many embodiments, the marker antibody is not labeled but in the course of an assay, it becomes indirectly labeled by binding to or being bound by another molecule that is labeled. The invention encompasses molecular complexes comprising a marker antibody and a label, as well as immunocomplexes comprising a marker polypeptide, a marker antibody, and immunocomplexes comprising a marker polypeptide, a marker antibody, and a label.

Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferones, fluoresceins, fluorescein isothiocyanate, rhodamines, dichlorotriazinylamine fluorescein, dansyl chloride, phycoelythrins, Alexa Fluor 647, Alexa Fluor 680, DilC₁₉(3), Rhodamine Red-X, Alexa Fluor 660, Alexa Fluor 546, Texas Red, YOYO-1+DNA, tetramethylrhodamine, Alexa Fluor 594, BODIPY FL, Alexa Fluor 488, Fluorescein, BODIPY TR, BODIPY TMR, carboxy SNARF-1, FM 1-43, Fura-2, Indo-1, Cascade Blue, NBD, DAPI, Alexa Fluor 350, aminomethylcoumarin, Lucifer yellow, Propidium iodide, or dansylamide; an example of a luminescent material includes luminol; examples of bioluminescent materials include green fluorescent proteins, modified green fluorescent proteins, luciferase, luciferin, and aequorin, and examples of suitable radioactive material include ¹²⁵I, ¹³¹I, ³⁵S or ³H. Many other examples of labels which can be used in the methods of the invention are disclosed in the following U.S. Pat. Nos. 4,774,339, 4,945,171, 5,132,432, 5,167,288, 5,227,487, 5,242,805, 5,248,782, 5,262,545, 5,274,113, 5,314,805, 5,316,906, 5,321,130, 5,326,692, 5,338,854, 5,362,628, 5,364,764, 5,405,975, 5,410,030, 5,433,896, 5,436,134, 5,437,980, 5,442,045, 5,443,986, 5,445,946, 5,451,663, 5,453,517, 5,459,268, 5,49,276, 5,501,980, 5,514,710, 5,516,864, 5,534,416, 5,545,535, 5,573,909, 5,576,424, 5,582,977, 5,616,502, 5,635,608, 5,635,608, 5,648,270, 5,656,449, 5,658,751, 5,686,261, 5,696,157, 5,719,031, 5,723,218, 5,773,227, 5,773,236, 5,786,219, 5,798,276, 5,830,912, 5,846,737, 5,863,753, 5,869,689, 5,872,243, 5,888,829, 6,005,113, 6,130,101, 6,162,931, 6,229,055, 6,265,179, 6,291,203, 6,31,267, 6,323,337, 6,329,205, 6,329,392 which are incorporated herein by reference in their entirety.

### 5.4 Differential Diagnosis

The present invention provides a variety of methods for determining a prognosis of B cell chronic lymphocytic leukemia (CLL) in a subject. In one embodiment, the methods of the invention can be used to determine whether the CLL is an indolent form of CLL or an aggressive form of CLL in a subject. In another embodiment, the methods can also be used to provide prognostic information for various aspects of CLL, and to supplement the mutation status of immunoglobulin heavy chain variable region gene and/or cytogenetics data for making a prognosis. The invention also provides methods to demonstrate correlation of certain aspects of CLL in a subject with the expression of the markers.

The subject who provides a sample may be but not limited to a member of a population that is the subject of a study in connection with CLL diagnosis or treatment, a subject suspected of having CLL, a patient diagnosed with CLL, an asyptomatic CLL patient, a symptomatic CLL, a CLL patient at any clinical stage as determined under the Rai system or Binet system, a CLL patient that has the indolent form of CLL, a CLL patient that has the aggressive form of CLL, or a patient with minimal residual CLL.

The subject may be a CLL patient that has a chromosome 13q deletion, chromosome 12 trisomy, a chromosome 6q deletion, a chromosome 11q deletion, or a chromosome 17p and/or p53 deletion. The subject may have been subjected to fluorescent in situ hybridization (FISH) test of their chromosomes, a determination of its IgV mutation status, and a diagnostic test based on the expression levels of CD38 and/or ZAP70. The methods of the invention can be used to confirm or supplement the results of these diagnostic tests. In addition to whether the prognosis is good (i.e, an indolent, slowly progressive disease) or poor (i.e., an aggressive disease that will in the near future becomes active), the methods of the invention may also be used to stratify a population by risk, to determine time to implement therapy in a patient without symptoms or in a patient with the indolent form of CLL, to predict survival over a period of time, or to predict remissions.

The subject may also be a CLL patient that has received treatment for the CLL, a CLL patient that is refractory to one type of CLL treatment, or a subject died of CLL with or without secondary complications. The methods can be applied to study whether expression of a marker in a subject is correlated with responsiveness to certain therapeutic modalities, or with the likelihood of becoming refractory to certain therapeutic modalities. The expression of individual markers or the marker expression profile in subjects classified by treatment histories and outcome can be analyzed to detect statistically significant correlations. It is contemplated that the marker expression profile of a subject can be used to aid selection of therapeutic modalities. Currently available treatment modalities include but is not limited to radiation, stem cell transplantation, gene therapy, immunotherapy and chemotherapy, for example, monotherapy with antimetabolites, e.g., fludarabine; anti-CD20 agents, e.g. rituximab (Rituxan, Genentech, Biogen Idec); or anti-CD52 agents, e.g., alemtuzumab (Campath, Berlex); or combination therapy with two or more of fludarabine, Rituxan, and/or Campath, autologous stem cell transplantation, allogenic stem cell transplantation, cord blood stem cell transplantation, heat shock protein-based vaccines (Antigenics), and bcl-2 antisense nucleic acid (Genasense, Genta).

The method of the invention comprises measuring at suitable time intervals before, during, or after therapy, the amount of marker gene product. Any change or absence of change in the amount of the marker gene product can be identified and correlated with the effect of the treatment on the subject. In one aspect, the method comprises determining the levels of marker gene product levels at different time points and to compare these values with a reference level. The reference level can be either the level of the marker present in normal, disease free individuals, individuals with characterized disease (indolent versus aggressive disease); and/or the levels present prior to treatment, or during remission of disease, or during periods of stability. These levels can then be correlated with the disease course, treatment outcome or overall survival.

The methods of the invention rely on the detection of the presence or absence of marker gene expression, or the qualitative or quantitative assessment of either over- or under-expression of marker gene in a population of test cells relative to a standard. Such methods utilize reagents such as marker polynucleotides and marker antibodies as described above.

### 5.4.1 Detection of Marker Nucleic Acid Molecules

Quantitative and qualitative aspects of marker gene expression can be assayed by many nucleic acid-based techniques well known in the art. For the detection of marker gene transcripts, ribonucleic acids from test cells are used as the starting point for such assay techniques, and may be isolated according to standard nucleic acid preparation procedures. The sample is a source of the test subject's ribonucleic acids which may include tissues, cells and biological fluids, and peripheral blood mononuclear cells, and purified CLL cells.

In other embodiments, an agent for detecting marker gene transcript is a labeled nucleic acid probe capable of hybridizing to marker mRNA, cRNA, or cDNA. The nucleic acid probe can be, for example, a full-length cDNA, such as the nucleic acid of SEQ ID NO: 1, 3, 5, or 7, or a portion thereof, such as a single-stranded oligonucleotide or nucleic acid of at least 15, 30, 50, 100, 250 or 500 contiguous nucleotides in length and sufficient to specifically hybridize under stringent conditions to the target marker polynucleotide.

Diagnostic methods for the detection of target marker polynucleotides molecules, in patient samples (such as B cells) or other appropriate cell sources, may involve hybridization assays and/or the amplification of specific gene sequences, e.g., by the polymerase chain reaction (PCR; see Mullis, K. B., 1987, U.S. Pat. No. 4,683,202). Many variations of these techniques are known in the art and can be applied in the methods of the invention.

In one embodiment of such a detection scheme, a cDNA molecule is synthesized from marker RNA molecules by reverse transcription. All or part of the resulting cDNA is then used as the template for a nucleic acid amplification reaction, such as PCR or the like. The nucleic acid reagents used as synthesis initiation reagents, i.e, the marker primers, in the reverse transcription and nucleic acid amplification steps of this method are chosen from among the marker polynucleotides described in Section 5.1. In other embodiments, the lengths of such single-stranded nucleic acid reagents are at least 9-30 nucleotides. For detection of the amplified product, the nucleic acid amplification may be performed using radioactively, fluorescently, luminescently, bioluminescently-labeled nucleotides.

In a one embodiment, the assays of the invention use quantitative PCR (QPCR) technology, see, for example, Bustin, S. A. (2002). “Quantification of mRNA using real-time reverse transcription PCR (RT-PCR): trends and problems.” J Molec Endocrin 29: 23-39; “Gene quantification using real-time quantitative PCR: An emerging technology hits the mainstream”, Ginzinger DG. Exp Hematol 2002 June; 30(6):503-12; “Quantitative RT-PCR: pitfalls and potential”, Freeman et al., (1999) Biotechniques 26, 112-122, which are incorporated herein by reference in their entirety.

In one embodiment, after the RNA is isolated from a sample, a marker specific reverse transcription (RT) reaction is performed, for example, in the same tube as the subsequent QPCR reaction, with a marker-specific primers. In another embodiment, total cDNA is generated from the RNA using random primers, oligo dT primers, or a combination of both. A portion of this cDNA is then used for QPCR reactions. With this method, cDNA from a single RT reaction can be used to analyze more than one marker genes. The amount of amplified marker polynucleotides is linked to fluorescence intensity using a fluorescent reporter molecule. The point at which the fluorescent signal is measured in order to calculate the initial template quantity can either be at the end of the reaction (endpoint QPCR) or while the amplification is still progressing (real-time QPCR). In endpoint QPCR, fluorescence data are collected after the amplification reaction has been completed, usually after 30-40 cycles, and this final fluorescence is used to back-calculate the amount of template present prior to PCR.

In other embodiments, the more sensitive and reproducible method of realtime QPCR is used to measure the fluorescence at each cycle as the amplification progresses. This allows quantification of the template to be based on the fluorescent signal during the exponential phase of amplification. A fluorescent reporter molecule (such as a double stranded DNA binding dye, or a dye labeled marker probe) is used to monitor the progress of the amplification reaction. The fluorescence intensity increases proportionally with each amplification cycle in response to the increased amplicon concentration, with QPCR instrument systems collecting data for each sample during each PCR cycle. The reporter molecule used in real-time reactions can be (1) a marker-specific probe composed of an oligonucleotide labeled with a fluorescent dye plus a quencher or (2) a non-specific DNA binding dye such as but not limited to SYBR®Green I that fluoresces when bound to double stranded DNA.

A higher level of detection specificity is provided by using an internal probe with primers to detect the QPCR product of interest. In the absence of a specific target sequence in the reaction, the fluorescent probe is not hybridized, remains quenched, and does not fluoresce. When the marker probe hybridizes to the target marker sequence, the reporter dye is no longer quenched, and fluorescence will be detected. The level of fluorescence detected is directly related to the amount of amplified target in each PCR cycle. A significant advantage of using probe chemistry compared to using DNA binding dyes is that multiple marker probes can be labeled with different reporter dyes and combined to allow detection of more than one target marker polynucleotide in a single reaction (multiplex QPCR).

For example, one approach for analyzing quantitative data is to use a standard curve that is prepared from a dilution series of control template of known concentration. A variety of sources can be used as standard templates including a plasmid containing a marker polynucleotide, genomic DNA, cDNA, in vitro transcripts, or total RNA.

RT-PCR techniques can also be utilized to detect differences in marker transcript size which may be due to normal or abnormal alternative splicing. Additionally, such techniques can be performed using standard techniques to detect quantitative differences between levels of full length and/or alternatively spliced marker transcripts detected in normal individuals relative to those individuals having cancer or exhibiting a predisposition toward neoplastic changes.

In the case where detection of specific alternatively spliced species or mutants is desired, appropriate primers and/or hybridization probes can be used, such that, in the absence of such sequence, no amplification would occur. Alternatively, primer pairs may be chosen utilizing the sequence data to choose primers which will yield fragments of differing size depending on whether a particular exon is present or absent from the marker transcript, or the choice of polyA signal being utilized.

As an alternative to amplification techniques, hybridization assays can be performed. Microarray-based assays can be used to detect and quantify the amount of marker gene transcript using cDNA- or oligonucleotide-based arrays. Microarray technology allows multiple marker gene transcripts and/or samples from different subjects to be analyzed in one reaction. Typically, mRNA isolated from a sample is converted into labeled nucleic acids by reverse transcription and optionally in vitro transcription (cDNAs or cRNAs labeled with, for example, Cy3 or Cy5 dyes) and hybridized in parallel to probes present on an array. See, for example, Schulze et al., Nature Cell Biol., 3 (2001), E190; and Klein et al., J Exp Med, 2001, 1625-1638, which are incorporated herein by reference in their entirety. Standard Northern analyses can be performed if a sufficient quantity of the test cells can be obtained. Utilizing such techniques, quantitative as well as size related differences between marker transcripts can also be detected.

Additionally, it is possible to perform such marker gene expression assays “in situ”, i.e., directly upon tissue sections (fixed and/or frozen) of patient cells or tissues, such that no nucleic acid purification is necessary.

The invention provides that when the amount of SEPT10 and/or Hs.23133 messenger RNA in a sample obtained from a composition comprising CLL cells, said sample being obtained from a test subject, is greater than the amount of SEPT10 and/or Hs.23133 messenger RNA in a similar sample obtained from a CLL patient or cell line displaying IgV mutations, the prognosis of the test subject is poor and the test subject likely has the aggressive form of CLL.

The invention also provides that when the amount of KIAA0799 and/or ADAM29 messenger RNA in a sample obtained from a composition comprising CLL cells, said sample being obtained from a test subject, is greater than the ratio observed in a similar composition of cells obtained from a CLL patient or cell line without IgV mutations, the prognosis of the test subject is good and the test subject likely has the indolent form of CLL.

The results obtained by the methods described herein may be combined with diagnostic test results based on other marker genes.

### 5.4.2 Detection of Marker Polypeptides

In another embodiment, the invention provides protein-based methods for detecting and measuring the levels of marker expression in a sample. Typically, the methods involve using detectably-labeled binding partners of marker polypeptides, such as anti-marker polypeptide-specific antibody, to bind marker polypeptides, variants or fragments thereof, or marker gene products (which are the result of alternatively spliced transcripts) in a sample. Many immunoassays known in the art can be applied. Such methods can also be used for studying abnormalities in the structure and/or temporal, tissue, cellular, or subcellular distribution of a marker polypeptide.

Depending on the assay technique applied, the sample may be processed prior to the assay. For example, the sample can be processed to enrich or purify a population of test cells, such as CLL cells, or to make the sample accessible to the reagents of the invention. In one embodiment, the target marker polypeptides are enriched or isolated from a tissue, whole cells, a cell extract or cell fraction. The protein isolation methods employed herein may, for example, be such as those described in Harlow and Lane (Harlow, E. and Lane, D., 1988, “Antibodies: A Laboratory Manual”, Cold Spring Harbor Laboratory Press, Cold Spring Harbor, N.Y.), which is incorporated herein by reference in its entirety.

In another embodiment, the marker polypeptides are detected in situ detection which may be accomplished by removing a cell sample or histological specimen from a patient, such as peripheral blood white blood cells and applying thereto a labeled antibody of the present invention. The antibody can be applied by overlaying the labeled antibody (or fragment) onto a sample. If the marker polypeptide is not present on the cell surface, it is preferable to introduce the antibody inside the cells, for example, by making the cell membrane permeable. Using the reagents of the invention, those of ordinary skill will readily perceive that any of a wide variety of flow cytometric methods and histological methods (such as staining procedures) can be modified in order to achieve such in situ detection.

Immunoassays for marker polypeptides will typically comprise incubating a sample, such as a biological fluid, a tissue extract, freshly harvested cells, or lysates of cells, in the presence of a detectably labeled antibody capable of identifying marker gene products or conserved variants or peptide fragments thereof, and detecting the bound antibody by any of a number of techniques well-known in the art.

One way of measuring the level of marker polypeptide with a specific marker antibody of the present invention is by enzyme immunoassay (EIA) such as an enzyme-linked immunosorbent assay (ELISA) (Voller, A. et al., J. Clin. Pathol. 31:507-520 (1978); Butler, J. E., Meth. Enzymol. 73:482-523 (1981); Maggio, E. (ed.), Enzyme Immunoassay, CRC Press, Boca Raton, Fla., 1980). The enzyme, either conjugated to the antibody or to a binding partner for the antibody, when later exposed to an appropriate substrate, will react with the substrate in such a manner as to produce a chemical moiety which can be detected, for example, by spectrophotometric, or fluorimetric means.

The biological sample may be brought in contact with and immobilized onto a solid phase support or carrier such as nitrocellulose, or other solid support which is capable of immobilizing cells, cell particles or soluble proteins. The support may then be washed with suitable buffers followed by treatment with the detectably labeled marker antibody. The solid phase support may then be washed with the buffer a second time to remove unbound antibody. The amount of bound label on solid support may then be detected by conventional means. A well-known example of such a technique is Western blotting.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. In one embodiment, supports include polystyrene beads. Those skilled in the art will know many other suitable carriers for binding antibody or antigen, or will be able to ascertain the same by use of routine experimentation.

A variety of immunoassay formats is available, which may be competitive or non-competitive, homogenous or heterogenous, and may include two-site or sandwich type assays, many of which are well-known in the art. Additional types of immunoassays include precipitin reactions, gel diffusion precipitin reactions, immunodiffusion assays, agglutination assays, complement fixation assays, radioimmunoassays, immunoradiometric assays, protein A immunoassays, and immunoelectrophoresis assays.

In another embodiment, flow cytometry is used to determine the level of marker gene expression in a population of test cells. Typically, the sample comprises cells expressing or suspected of expressing a marker, such as CLL cells. The cells in a sample are contacted with a marker antibody which may be labeled with a fluorochrome. Alternatively, the cells can be indirectly labeled, i.e., after contact with a marker antibody, the cells are stained with a fluorochrome-labeled secondary antibody or fluorochrome-labeled reagents. For marker polypeptides present in intracellular locations, the cells are permeabilized by techniques known in the art. In other embodiments, the test cells are enriched for CLL cells which can be stained by fluorochrome-labeled antibodies to CD5, CD19, and/or CD23, and sorted or identified by flow cytometric techniques in a separate protocol or in the same protocol. Accordingly, the test cells can be CD5+ cells, CD19+ cells, CD23+ cells, CD5+/CD19+ cells, CD5+/CD23+ cells, CD23+/CD19+ cells, or CD5+/CD19+/CD23+ cells. For those markers which have a restricted pattern of expression (e.g., SEPT10 the expression of which is negligible in normal B and T cells), it is possible to use whole blood cells or cell subpopulations which have not been subjected to multiple enrichment or purification steps. Accordingly, the methods comprise detecting a deviation in the number of test cells expressing a marker polypeptide in a sample from the patient, relative to a reference sample number. In one embodiment, the ratio of test cells expressing a marker in a defined cell population from a subject is determined, and compared to a reference ratio for the same defined cell population.

The invention provides that when the ratio of cells that are expressing SEPT10 and/or Hs.23133 in a composition comprising CLL cells obtained from a test subject is greater than the ratio observed in a similar composition of cells obtained from a CLL patient or cell line displaying IgV mutations, the prognosis of the test subject is poor and the test subject likely has the aggressive form of CLL.

The invention also provides that when the ratio of cells that are expressing KIAA0799 and/or ADAM29 in a composition comprising CLL cells obtained from a test subject is greater than the ratio observed in a similar composition of cells obtained from a CLL patient or cell line without IgV mutations, the prognosis of the test subject is good and the test subject likely has the indolent form of CLL.

The binding activity of a given lot of marker antibody or CLL antigen antibody may be determined according to well known methods. Those skilled in the art will be able to determine operative and optimal assay conditions including internal controls for each determination by employing routine experimentation.

### 5.5 Kits

The present invention also provides kits for practicing the methods of the invention. The kits can be used for clinical diagnosis and/or laboratory research. In one embodiment, a kit comprises one or more diagnostic reagents in one or more containers. In another embodiment, the kit also comprises instructions in any tangible medium on the use of the diagnostic reagent(s) in one or more methods of the invention.

For nucleic acid-based methods, such as hybridization assays or polymerase chain reaction, a diagnostic reagent in the kit may comprise at least one of the following: marker polynucleotide, marker probe, and/or marker primer. The diagnostic reagents may be labeled, for example, by one or more different fluorochromes. Such a kit may optionally provide in separate containers enzymes and/or buffers for reverse transcription, in vitro transcription, and/or DNA polymerization, nucleotides, and/or labeled nucleotides, including fluorochrome-labeled nucleotides. Also included in the kit may be positive and negative controls for the methods of the invention.

For protein-based methods, such as flow cytometry, a diagnostic reagent in the kit may comprise a marker antibody, which may be labeled, for example, by a fluorochrome. Such a kit may optionally provide in separate containers buffers, secondary antibodies, signal generating accessory molecules, labeled secondary antibodies, including fluorochrome-labeled secondary antibodies. The kit may also include unlabeled or labeled antibodies to various cell surface antigens which can used for identification or sorting of subpopulations of cells, e.g., anti-CD5 antibodies, anti-CD19 antibodies, and the like. Also included in the kit may be positive and negative controls for the methods of the invention.

The positive and/or negative controls included in a kit can be nucleic acids, polypeptides, cell lysate, cell extract, whole cells from patients, or whole cells from cell lines, that are of a known genotype/phenotype.

The following examples illustrate the present invention, and are set forth to aid in the understanding of the invention, and should not be construed to limit in any way the scope of the invention as defined in the statements of the invention which follow thereafter.

### 6. EXAMPLES

### Differential Expression of Marker Genes

This example demonstrates the differential expression of marker genes in two groups of CLL patients—those displaying IgV mutations (“mutated CLL”) and, those without IgV mutations (“unmutated CLL”).

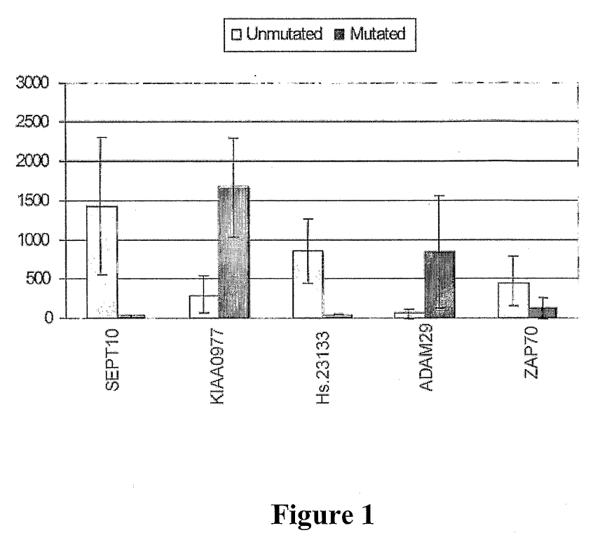

FIG. 1 shows the expression values of five transcripts that upon microarray analysis and other considerations were found to be the best candidates to discriminate between unmutated and mutated CLL cases. Of the five transcripts, three, including ZAP70, exhibited higher expression in unmutated CLL. There was variation of expression of respective transcripts within the two subtypes, especially noticeable for ZAP70. Less overlap in expression was seen for SEPT10, KIAA0977, and Hs.23133. This comparison shows that these three transcripts represent better candidates to discriminate unmutated from mutated CLL than ZAP70.

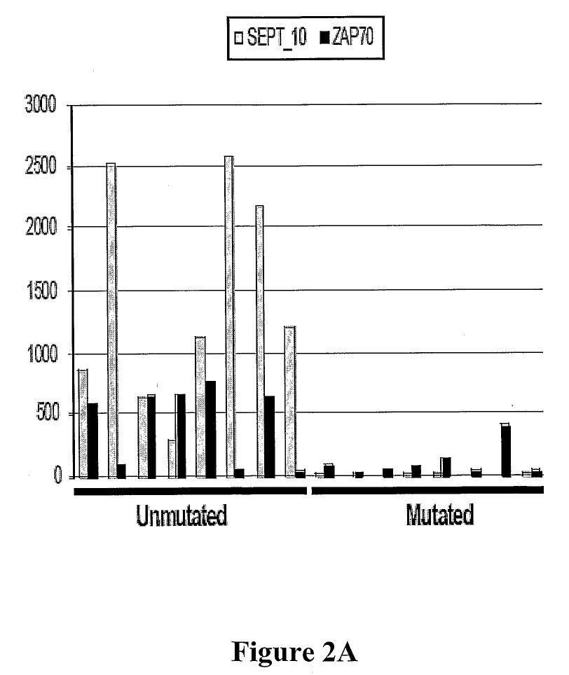

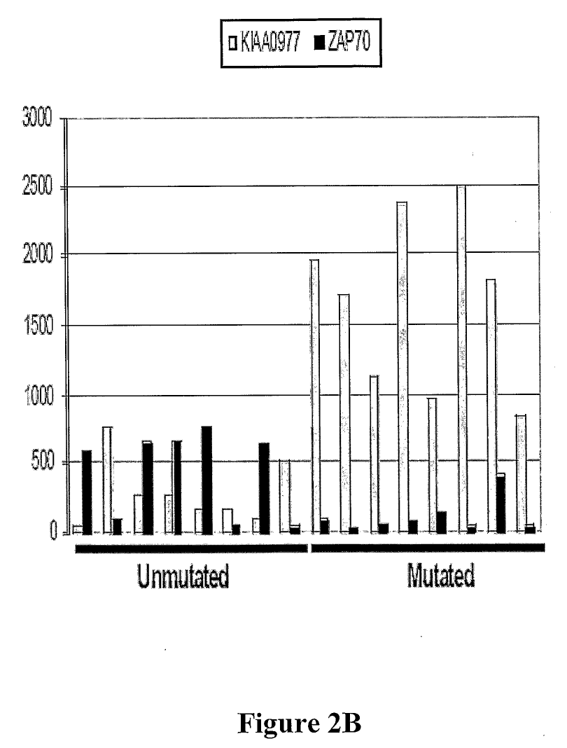

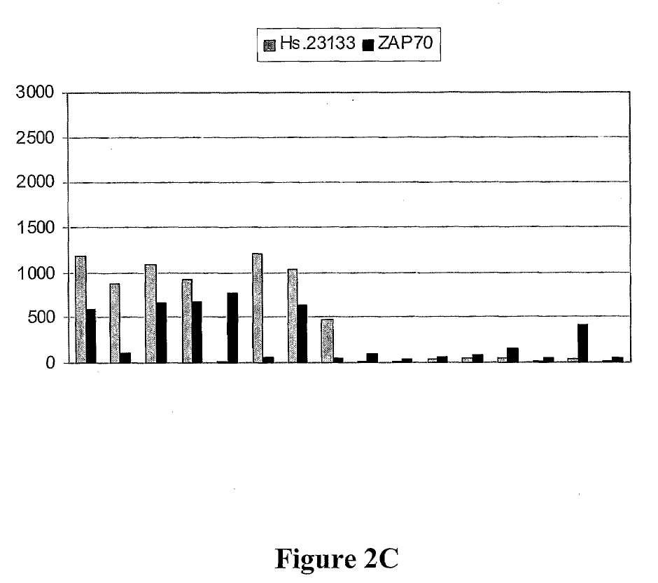

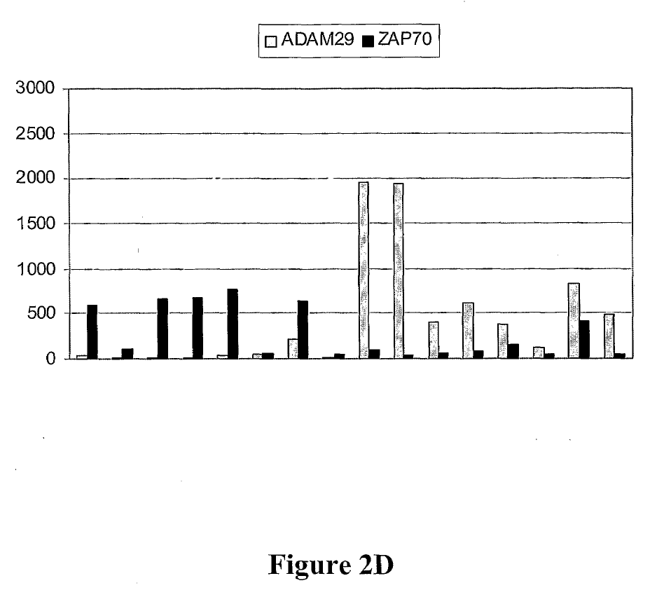

Examination of the expression levels of SEPT10, KIAA0977, Hs.23133, ADAM29 and ZAP70, in individual CLL cases are shown in FIGS. 2A, 2B, 2C and 2D. Firstly, it is noted that for ZAP70, there is some overlap in the expression levels between mutated and unmutated CLL, consistent with the 10% discordance reported for the flow cytometric analysis of ZAP-70 versus IgV mutational status (Orchard et al., ZAP-70 expression and prognosis in chronic lymphocytic leukemia. Lancet 363:105-111, 2004.). The results confirm that the expression pattern of all four markers can be correlated with IgV mutation status in individual CLL cases. For KIAA0977, the overlap in expression between unmutated and mutated CLL is less apparent. SEPT10 represents the best candidate due to almost complete lack of detectable expression noted in mutated CLL, with no overlap in expression with unmutated CLL.

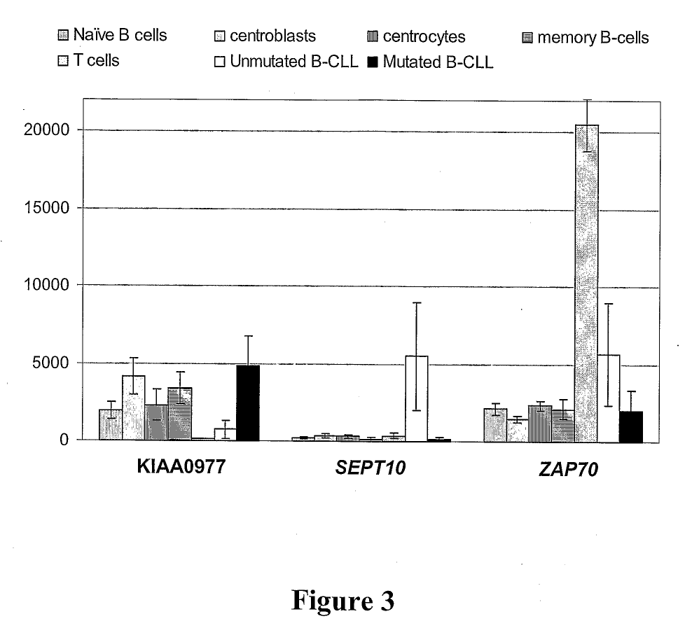

ZAP-70 is normally expressed in T and natural killer (NK) cells, and thus requires careful gating during FACS analysis of all specimens. The accuracy of using ZAP70 can be questionable in evaluating CLL specimens with high T cell counts. To further demonstrate that SEPT10 and KIAA0977 are better candidates, the expression of these two transcripts as well as ZAP70, in purified subsets of lymphoid cells were studied. As expected, marked expression of ZAP70 was detected in T cells, with lower levels detected in all other B cell subtypes examined (FIG. 3). The expression levels of KIAA0977 in mutated CLL cells were comparable to those exhibited by normal B cells, thus making this marker more suitable for flow cytometric analysis. SEPT10 expression was barely detected in normal B and T cells, and mutated CLL cells. Based on these data, SEPT10 represents a better surrogate marker for mutational status in CLL than ZAP70.

### 7. EXAMPLES

### SEPT10 Flow Cytometry

This example illustrate the establishment of a flow cytometric assay for the evaluation of Septin 10 expression in CLL and to demonstrate the relationship between Septin 10 expression and immunoglobulin heavy chain V region (IGHV) mutational status. With minor modifications that will be apparent to one of skill in the art, the same approach can be adapted to use KIAA0977, Hs.23133 and/or ADAM29 antibodies in flow cytometric assays in the assay methods of the invention.

### 7.1 Materials and Methods

Cell Lines, Patients, and Specimens Human cell lines, whole blood and PBMC specimens from both normal healthy donors and CLL patients is utilized. A panel of human tumor cell lines is used which includes: 697 (pre-B cell), CB33 and Ramos (mature B cells), U266 and SKMM1 (plasma B cells), Jurkat (T cells), K562 (chronic myelogenous leukemia) and HeLa S3 (cervical cancer). The cell lines are routinely maintained by standard methods. Blood is obtained from both healthy donors and CLL patients from multiple medical centers on a payment per specimen basis, with written consent for use by the patient. Ten normal bloods and 50 bloods from patients with a diagnosis of CLL based on standard morphological and immunophenotypical criteria are used. Blood samples are coded to maintain anonymity.

Specimen Handling. For the assays, blood samples are handled as follows: Normal blood (10 cases): Peripheral Blood Mononuclear Cells (PBMC) are isolated on Ficoll-Hypaque gradients with a portion used fresh for cytometric analysis and another frozen in dimethylsulphoxide (DMSO). In few cases, fresh whole blood is also utilized for flow cytometric analysis. CLL blood (50 cases): PBMC are isolated and used as described above. In five cases, fresh whole blood is utilized directly for flow cytometric analysis.

For all specimens except whole blood targeted directly for flow cytometric analysis, the separated PBNC are portioned out for flow cytometric assays and DNA extraction, and in those cases with adequate cells, for RNA extraction and lysate preparation. For flow cytometric analysis, cells are fixed in paraformaldehyde and permeabilized with Tween according to standard procedures. DNA and RNA are extracted, quantitated, and evaluated for quality and cellular lysates are prepared and protein concentration estimated as described in Houldsworth et al., Cell Growth Differ. 8:293-299 (1997).

IGHV Mutation Analysis. DNA extracted from all CLL and normal PBMC are subjected to sequence analysis of the IGHV genes as described in Pasqualucci et al., Cancer Res 60:5644-5648, (2000). Briefly, the DNA is amplified by PCR using a set of six VH family-specific primers annealing to sequences in the framework region I in separate reactions, along with a JH primer mix. PCR is performed for 34 cycles, with aliquots run on ethidium bromide-stained 2% agarose gels. In the case of amplification failure, the sense primers are replaced with oligonucleotides complementary to the leader sequences of the VH genes. PCR products are purified (Qiagen) and sequenced directly from both strands using the Big Dye Terminator Cycle System (ABI) with an ABI automated DNA sequencer. Sequence analysis and alignments are performed with the use of the IMGT database and sequence alignment tool (http://www.ebi.ac.uk/imgt). Specimens with fewer than 2 percent of base pairs differing from those of the consensus sequence are considered unmutated, according to current conventions.

### 7.2 Generation of Septin 10 Antibody and Western Blotting

Rabbit polyclonal and murine monoclonal antibodies directed against human Septin 10 are generated by a commercial source (ProSci Inc.) using recombinant Septin 10 protein and/or synthesized peptides that are predicted to represent the best antigens. Both sets of antibodies are tested for specificity by Western blotting of 293 cells transfected with a Septin 10-expression vector and by blocking with antigenic peptides. SEPT10 was originally identified in dendritic cells, but reported to be expressed in a variety of human tissues and tumor cell lines including HeLa S3 and K562 cells, by Northern blotting. Expression is barely detectable in PBMC. Septin 10 expression is evaluated by Western blotting of a panel of B cell lines corresponding to different stages of B cell differentiation (Chen et al., Blood 91:603-607, (1998)) where PBMC from healthy donors and HeLa S3 and K562 cell lines will serve as negative and positive controls respectively. This analysis provides an evaluation of non-specific interactions by the individual antibodies generated. A B cell line found not to be expressing Septin 10 is mixed with increasing percentage of healthy donor PBMC in order to determine if the presence of other mononuclear cells influences the level of expression of Septin 10. No influence is reported which is consistent with the lack of expression at the RNA level in other normal B and T cells. Lysates from CLL cells are evaluated for expression of Septin 10 by Western blotting, permitting a semiquantitative correlation between expression and mutational status.

Antibody Generation Polyclonal and monoclonal antibodies for Septin 10 are generated, for example, using recombinant Septin 10 protein. Recombinant Septin 10 is produced using a baculovirus expression system. A baculoviral vector is constructed to contain a full-length coding sequence for SEPT10 cDNA and following Sf9 insect cell transfection, cells are collected and extracted according to standard procedures. The antibodies are generated by ProSci Inc. As an alternative, antigenic peptides are designed and synthesized by ProSci Inc. for antibody production.

Western Blotting Preparation of cell lysates are prepared from the cell lines and PBMC, and western blotting performed as described in Houldsworth et al., Cell Growth Differ. 8:293-299 (1997). The primary antibodies are the polyclonal and monoclonal anti-Septin 10 antibodies developed as described above, and murine anti-chicken α-tubulin (Calbiochem) is a control for loading. 293 cells transfected with an expression vector containing a full-length SEPT10 cDNA will be generated by standard techniques, and used in the testing for antibody specificity as described above.

### 7.3 Flow Cytometric Assay for Septin 10

A flow cytometric assay for Septin 10 expression in CLL cells is used on PBMC and whole blood. Based on the RNA expression pattern noted for SEPT10 few if any other normal PBMC can influence the overall expression levels, indicating a robust single staining procedure. Initially, PBMC (fresh and DMSO-frozen) from blood from healthy individuals and CLL patients are evaluated for Septin 10, CD19, and CD5 expression by flow cytometry. The range of Septin 10 expression on CD19+ B cells in PBMC from normal donors is determined, and compared between CD5+ and CD5− B cell subpopulations. A similar analysis is performed on few whole blood specimens that are initially gated according to initial side and forward scatter plots on lymphocytes. For CLL PBMC, the percentage of Septin 10-positive cells is analyzed both as a percentage of CD19+/CD5+ CLL cells and of cells in the lymphocyte gate. Likewise for few whole blood CLL specimens, after gating of lymphocytes, the percentage of Septin 10-staining cells will be determined. Along with the percentages noted for normal donors, comparison between expression levels obtained in the lymphocyte gate and to CD19+/CD5+ cells indicates the more reliable of the two evaluations, and additionally permit evaluation of the potential use of a single staining procedure for Septin 10 expression. In this manner, the percentage of Septin 10-staining cells in all normal and CLL specimens are obtained for determining a prognosis for CLL.

The lack of expression of Septin 10 in normal B and T cells permit a direct determination of the percentage of Septin 10-staining cells in specimens. This is unlike ZAP-70, where normal T cells exhibited marked expression influencing ZAP-70 positivity in cases with high T cell counts. A routine double or triple staining assay for Septin-10 with one of or both CD19 and CD5 as described above may be used as controls for the performance of the assy.

After fixation and permeabilization as described above, PBMC are incubated with anti-Septin 10 antibody, followed by a fluorescein isothiocyanate (FITC)-conjugated secondary antibody. The cells are incubated with anti-CD5-allophycocyanine and anti-CD19-peridinin chlorophyll protein cychrome 5.5 antibodies (both from BD Biosciences). For example, approximately 10,000 CD19+/CD5+ cells are acquired, and a FACS Calibur flow cytometer using CellQuest software (both from BD Biosciences) with gating according to side and forward scatter plots to exclude inclusion of debris, monocytes, and doublets. Septin 10-positive cells are calculated as a percentage of cells in the lymphocyte gate and after additional gating of CD19+/CD5+ cells. Appropriate isotype controls are performed in all cases.

### 7.4 Septin 10 expression as a surrogate for IGHV mutational Status

Correlative analyses is undertaken to demonstrate the expression of Septin 10 in the flow cytometric assay as a surrogate marker for IGHV mutational status.

First, the level of SEPT10 RNA levels is compared to Septin 10 expression level by Western blotting and flow cytometry. The relative levels of the SEPT10 transcript are determined by semiquantitative RT-PCR and compared with the percentage of cells expressing Septin 10 by flow cytometry and/or relative levels by Western blotting. Second, a cut-off in the percentage of cells staining for Septin 10 is established to distinguish high versus low expressers. The cut-off is determined using a receiver-operating-characteristic plot (Fisher L D, Van Belle G. Biostatistics. Wiley (New York), 1993), which will show the relationship between sensitivity and specificity as a function of the cut-off. The statistical correlation between Septin 10 expression (high or low) and mutational status (mutated or unmutated) is performed using Fisher's exact test. Since choosing an optimal cut-off will give overly optimistic estimates of sensitivity and specificity, adjustments are made to these estimates using the method of cross-validation.

Only two other markers have been reported as surrogates for IGHV mutational status: CD38 and ZAP-70. For CD38, correlation was not confirmed in additional studies, and ZAP-70 has yet to undergo evaluation. Thus, in the present application, the expression of ZAP-70 was examined in the present panel of CLL specimens as described by Crespo et al., New Engl J Med 348:1764-1775, 2003, and Orchard et al., Lancet 363:105-111, 2004, and correlative analysis with mutational status was carried out as described above for Septin 10. McNemar's test based on different cut-offs of sensitivity and specificity will indicate whether Septin 10 or ZAP-70 expression is significantly different as a surrogate for IGHV mutational status in CLL (Fisher L D, Van Belle G. Biostatistics. Wiley (New York), 1993).

RT-PCR: First strand cDNA synthesis is performed on RNA isolated from normal and CLL PBMCs as described. Multiplex PCR contain forward and reverse primers with SEPT10 and ACTB-specific primers, the latter for quantitation purposes (Bourdonet al., Cancer Res 62:6218-6223, 2002). The Kendall's tau rank correlation coefficient (Fisher L D, Van Belle G. Biostatistics. Wiley (New York), 1993) is used to evaluate the relationship between Septin 10-staining cells (percentage of cells) or Septin 10/U-tubulin levels, and SEPT10 transcript levels (SEPT10/ACTB ratio).

Flow cytometry: For ZAP-70 expression by normal and CLL PBMC, an anti-human ZAP-70 murine monoclonal antibody (Upstate Biotechnology) is used, and after incubation with an appropriate FITC-conjugated secondary antibody, the cells are also be incubated with CD19-peridinin chlorophyll protein cychrome 5.5, CD5-allophycocyanine, CD3-phycoerythrin, and CD56-phycoerythrin (all from BD Biosciences). Gating and calculation of percentage ZAP-70-staining cells are performed as described in Crespo et al., and Orchard et al. cited above.

All references and database records for the GenBank accession numbers cited herein are incorporated herein by reference in their entirety and for all purposes to the same extent as if each individual publication, record, or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended statements of the invention along with the full scope of equivalents to which such statements are entitled.

1. A method for determining a prognosis for B cell chronic lymphocytic leukemia (CLL) in a subject, said method comprising:
(a) determining a level of expression of at least one marker gene in test cells of a subject, wherein said at least one marker gene is SEPT10, KIAA0799, Hs.23133, or ADAM29; and
(b) determining said prognosis for said subject based on the level of expression of said at least one marker gene in said test cells, wherein a high level of expression of SEPT10 relative to a reference SEPT 10 level indicates a prognosis of aggressive CLL; a high level of expression of Hs.23133 relative to a reference Hs.23133 level indicates a prognosis of aggressive CLL; a low level of expression of KIAA0799 relative to a reference KIAA0799 level indicates a prognosis of indolent CLL; and a low level of expression of ADAM29 relative to a reference ADAM29 level indicates a prognosis of indolent CLL.

2. The method of claim 1, wherein the reference SEPT10 level, reference KIAA0799 level, reference Hs.23133 level, reference ADAM29 level, or any combination thereof are established from a clinically-characterized population of patients.

3. The method of claim 2, wherein the clinically-characterized population of patients display mutations in the genes encoding immunoglobulin heavy chain variable regions.

4. The method of claim 1, wherein said test cells comprise chronic lymphocytic leukemia cells, CD5+/CD19+/CD23+ cells, CD5+/CD19+ cells, CD19+/CD23+ cells, CD5+/CD23+ cells, B cells, or any combination thereof.

5. The method of claim 1, wherein said test cells are peripheral mononuclear blood cells, or whole blood cells.

6. The method of claim 1, wherein the level of expression of said at least one marker gene is determined by measuring an amount of at least one marker polypeptide expressed in said test cells.

7. The method of claim 1, wherein the level of expression of said at least one marker gene is determined by measuring a ratio of test cells expressing said at least one marker gene in a batch of test cells relative to the total number of test cells in said batch of test cells.

8. The method of claim 7, wherein the ratio of test cells expressing said at least one marker in said batch of test cells relative to the total number of test cells in said batch of test cells is measured by flow cytometry.

9. The method of claim 1, wherein the level of expression of said at least one marker gene is determined by measuring an amount of marker messenger RNA.

10. The method of claim 9, wherein the amount of marker messenger RNA is measured by DNA-DNA hybridization, RNA-DNA hybridization, reverse transcription-polymerase chain reaction.

11. A diagnostic reagent comprising a fluorochrome-labeled anti-SEPT 10 antibody, a fluorochrome-labeled anti-KIAA0799 antibody, a fluorochrome-labeled anti-Hs.23133 antibody, or a fluorochrome-labeled anti-ADAM29 antibody.

12. A diagnostic reagent comprising (a) anti-CD5 antibody, anti-CD19 antibody, anti-CD23 antibody, or any combination thereof; and (b) anti-SEPT 10 antibody, anti-KIAA0799 antibody, anti-Hs.23133 antibody, anti-ADAM29 antibody, or any combination thereof.

13. A diagnostic composition comprising (a) anti-SEPT10 antibody, anti-KIAA0799 antibody, anti-Hs.23133 antibody, anti-ADAM29 antibody, or any combination thereof; and (b) test cells comprising chronic lymphocytic leukemia cells, CD5+/CD 19+/CD23+ cells, CD5+/CD19+ cells, CD19+/CD23+ cells, CD5+/CD23+ cells, B cells, or any combination thereof, said test cells being obtained from a human in need of a prognosis of chronic lymphocytic leukemia.

14. A diagnostic composition comprising (a) SEPT10 polynucleotides, KIAA0799 polynucleotides, Hs.23133 polynucleotides, anti-ADAM29 polynucleotides, or any combination thereof; and (b) nucleic acids obtained from test cells of a human in need of a prognosis of chronic lymphocytic leukemia.

15. A test kit comprising a diagnostic reagent comprising anti-SEPT10 antibody, anti-KIAA0799 antibody, anti-Hs.23133 antibody, anti-ADAM29 antibody, or any combination thereof; and instructions for using said diagnostic reagent in providing a prognosis of chronic lymphocytic leukemia.

16. An antibody to Hs.23133 polyeptide.
